(19) United States
(12) Patent Application Publication
(10) Pub. No.: US 2025/0295741 A1
(43) Pub. Date: Sep. 25, 2025

(54) PREVENTION OF POST-OPERATIVE ATRIAL FIBRILLATION WITH A BOTULINUM TOXIN

(71) Applicant: Allergan, Inc., North Chicago, IL (US)

(72) Inventors: William G. Ferguson, Easton, CT (US); Alexandra T. O'sullivan, Staten Island, NY (US); Richard Deveson Leaback, Teddington (GB); David B. Bharucha, North Chicago, IL (US)

(21) Appl. No.: 19/126,369

(22) PCT Filed: Nov. 7, 2023
(86) PCT No.: PCT/US2023/036902

§ 371 Date: May 1, 2025

Related U.S. Application Data
(60) Provisional application No. 63/423,165, filed on Nov. 7, 2022.

(51) Int. Cl. A61K 38/48; A61P 9/06

(52) U.S. Cl. CPC A61K 38/4893; A61P 9/06; C12Y 304/24069

(57) ABSTRACT

Methods are provided for preventing post-operative atrial fibrillation by administering a botulinum toxin to epicardial fat pads in the heart of a patient.

### RELATED APPLICATIONS

This application claims priority to U.S. Provisional Application No. 63/423,165, filed on Nov. 7, 2022. The entire contents of the foregoing application are expressly incorporated herein by reference.

### TECHNICAL FIELD

The subject matter described herein relates to a method for preventing post-operative atrial fibrillation by administering a botulinum toxin into epicardial fat pads in distinct regions of the heart of a patient.

### BACKGROUND

Atrial fibrillation (AF) is a common complication following cardiac surgery. Post-operative AF (POAF) affects between 30 and 60% of patients undergoing cardiac surgery and the incidence rate has remained relatively constant over the last several decades. More specifically, rates of POAF increase with the complexity of cardiac surgery, with up to 40% of patients undergoing isolated coronary artery bypass grafting (CABG) surgery experiencing POAF and even higher rates in patients undergoing valve and valve plus CABG surgery.

POAF can worsen a patient's hemodynamic status during the vulnerable post-surgical period. It also increases risk of hypotension, heart failure, stroke, and death. POAF can often necessitate additional pharmacologic therapy, including medications for rate or rhythm control or stroke prevention, or procedures such as cardioversion. Drug therapies carry risks, including increased risks of bleeding with oral anticoagulation and increased risks of symptomatic bradycardia with atrioventricular nodal blocking agents, such as amiodarone.

In addition to the increased risks of morbidity and mortality to patients, POAF is associated with a significant increase in healthcare utilization and cost. Data from the Society for Thoracic Surgeons registry has shown that POAF is independently associated with a 25% increased length of stay. The increased healthcare utilization due to POAF has been associated with a 23% increase in hospital costs. The burdens imposed by POAF on both patients and healthcare systems has led to an increase in research focused on its prevention.

Despite an increasing number of studies focused on prevention of POAF over the past two decades, the number of effective therapies with a class I recommendation in the current clinical guidelines (Journal of the American College of Cardiology Vol. 64, No. 21, 2-14) is limited. Perioperative beta-blocker therapy is effective and is recommended for the prevention of POAF, but only 70% of patients receive beta-blockers and many need to have the therapy withdrawn post-operatively. An effective medication for the prevention of POAF is amiodarone; however, it is used in only 1 in 10 patients undergoing cardiac surgery due to its potential for adverse events, including bradycardia and organ toxicity. Recommendations for amiodarone therapy are focused on patients with higher risk for POAF and it is not recommended in all patients undergoing cardiac surgery. Despite guideline recommendations for beta-blocker therapy and amiodarone in patients at elevated risk, currently, there are no drugs approved by the Food and Drug Administration or the European Medicines Agency specifically for the prevention of POAF.

There are several other therapies where evidence supporting their use is not clear or is not well-established, including but not limited to sotalol, biatrial pacing, post-operative colchicine, and post-operative steroids. In small trials, statin therapy showed promise for the prevention of POAF, but a large placebo-controlled randomized trial in 1,922 patients found no evidence of reduction in POAF with rosuvastatin. Thus, there is a clear and unmet need for additional medical interventions that are safe and effective for the prevention of POAF.

### BRIEF SUMMARY

The following aspects and embodiments thereof described and illustrated below are meant to be exemplary and illustrative, not limiting in scope.

A method for preventing or attenuating risk of POAF by administering a neuromodulating substance into epicardial fat pads is provided. The method is beneficial in that it can be performed with a minimal increase in cardiopulmonary bypass time and/or cardioplegia time. The method is an intra-operative cardiac neuromodulation with a minimal impact on operating time, thus minimizing the need for manipulation of the heart during the procedure and avoiding permanent destruction of cardiac and cardiac-neural tissue that occurs with ablation procedures.

In one aspect, provided herein is a method for preventing post-operative atrial fibrillation (POAF) in a subject in need thereof, comprising (1) selecting a subject in need of open-chest, coronary artery bypass grafting (CABG) surgery, and (2) administering to five epicardial fat pads of the subject about 125 units of a botulinum toxin serotype A by injection, distributed across the five epicardial fat pads, while the subject is undergoing open-chest CABG surgery, wherein the five epicardial fat pads comprise an aortic fat pad, a superior right-side pulmonary vein fat pad, an anterior right-side pulmonary vein fat pad, a superior left-side pulmonary vein fat pad, and an anterior left-side pulmonary vein fat pad, wherein the subject does not exhibit at least one continuous post-operative atrial fibrillation (POAF) episode having a duration of 30 seconds or more during a period of 30 days from administration, thereby preventing post-operative atrial fibrillation in the subject.

In some embodiments, the method further comprises selecting a subject that is about 65 years of age or older. In some embodiments, the method further comprises selecting a subject that is not in need of valve surgery. In some embodiments, the method does not comprise valve surgery during the open-chest CABG surgery.

In some embodiments, during a period of 30 days from administration, the subject does not exhibit at least one continuous post-operative atrial fibrillation episode having a duration selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours.

In some embodiments, during a period of 30 days from administration, the subject does not exhibit at least one continuous post-operative atrial fibrillation episode having a duration equal to or greater than 5 minutes.

In some embodiments, during a period of 30 days from administration, the subject does not exhibit at least one continuous post-operative atrial fibrillation episode having a duration equal to or greater than 30 minutes.

In some embodiments, during a period of 30 days from administration, the subject does not exhibit at least one continuous post-operative atrial fibrillation episode having a duration equal to or greater than 1 hour.

In some embodiments, during a period of 30 days from administration, the subject does not exhibit at least one continuous post-operative atrial fibrillation episode having a duration equal to or greater than 4 hours.

In some embodiments, during a period of 30 days from administration, the subject does not exhibit at least one continuous post-operative atrial fibrillation episode having a duration equal to or greater than 30 seconds, equal to or greater than 30 minutes, equal to or greater than 4 hours, or combinations thereof. In some embodiments, during a period of 30 days from administration, the subject does not exhibit at least one continuous post-operative atrial fibrillation episode having a duration equal to or greater than 30 seconds, equal to or greater than 2 minutes, equal to or greater than 5 minutes, or combinations thereof. In some embodiments, during a period of 30 days from administration, the subject does not exhibit at least one continuous post-operative atrial fibrillation episode having a duration equal to or greater than 30 seconds, equal to or greater than 1 hour, and equal to or greater than 6 hours, or combinations thereof.

In some embodiments, administering the botulinum toxin serotype A to the subject reduces the occurrence or likelihood of one or more post-operative atrial fibrillation episodes by at least 20% during a period of 30 days from administration, as compared to a subject that does not receive administration of the toxin serotype A while undergoing open-chest CABG surgery, and wherein the one or more atrial fibrillation episodes have a duration of 30 seconds or more.

In some embodiments, the one or more post-operative atrial fibrillation episodes have a duration selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours during a period of 30 days from administration. In some embodiments, the one or more post-operative atrial fibrillation episodes have a duration of equal to or greater than 30 seconds. In some embodiments, the one or more post-operative atrial fibrillation episodes have a duration of equal to or greater than 5 minutes. In some embodiments, the one or more post-operative atrial fibrillation episodes have a duration of equal to or greater than 30 minutes. In some embodiments, the one or more post-operative atrial fibrillation episodes have a duration of equal to or greater than 1 hour. In some embodiments, the one or more post-operative atrial fibrillation episodes have a duration of equal to or greater than 4 hours.

In some embodiments, the one or more post-operative atrial fibrillation episodes have a duration of equal to or greater than 30 seconds, equal to or greater than 30 minutes, equal or greater than 4 hours, or combinations thereof. In some embodiments, the one or more post-operative atrial fibrillation episodes have a duration equal to or greater than 30 seconds, equal to or greater than 2 minutes, equal to or greater than 5 minutes, or combinations thereof. In some embodiments, the one or more post-operative atrial fibrillation episodes have a duration equal to or greater than 30 seconds, equal to or greater than 1 hour, and equal to or greater than 6 hours, or combinations thereof.

In some embodiments, administering the botulinum toxin serotype A to the subject reduces the occurrence or likelihood of one or more post-operative atrial fibrillation episodes by at least 30% during a period of 30 days from administration, as compared to a subject that does not receive administration of the toxin serotype A while undergoing open-chest CABG surgery, and wherein the one or more atrial fibrillation episodes have a duration of 30 seconds or more.

In some embodiments the one or more post-operative atrial fibrillation episodes have a duration selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours during a period of 30 days from administration. In some embodiments the one or more post-operative atrial fibrillation episodes have a duration of equal to or greater than 30 seconds. In some embodiments the one or more post-operative atrial fibrillation episodes have a duration of equal to or greater than 5 minutes. In some embodiments the one or more post-operative atrial fibrillation episodes have a duration of equal to or greater than 30 minutes. In some embodiments the one or more post-operative atrial fibrillation episodes have a duration of equal to or greater than 1 hour. In some embodiments the one or more post-operative atrial fibrillation episodes have a duration of equal to or greater than 4 hours.

In some embodiments, the one or more post-operative atrial fibrillation episodes have a duration of equal to or greater than 30 seconds, equal to or greater than 30 minutes, equal or greater than 4 hours, or combinations thereof. In some embodiments, the one or more post-operative atrial fibrillation episodes have a duration equal to or greater than 30 seconds, equal to or greater than 2 minutes, equal to or greater than 5 minutes, or combinations thereof. In some embodiments, the one or more post-operative atrial fibrillation episodes have a duration equal to or greater than 30 seconds, equal to or greater than 1 hour, and equal to or greater than 6 hours, or combinations thereof.

In some embodiments, administering the botulinum toxin serotype A to the subject reduces the occurrence of one or more post-operative atrial fibrillation episodes by at least 40% during a period of 30 days from administration, as compared to a subject that does not receive administration of the toxin serotype A while undergoing open-chest CABG surgery, and wherein the one or more atrial fibrillation episodes have a duration of 30 seconds or more.

In some embodiments the one or more post-operative atrial fibrillation episodes have a duration selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours during a period of 30 days from administration. In some embodiments the one or more post-operative atrial fibrillation episodes have a duration of equal to or greater than 30 seconds. In some embodiments the one or more post-operative atrial fibrillation episodes have a duration of equal to or greater than 5 minutes. In some embodiments the one or more post-operative atrial fibrillation episodes have a duration of equal to or greater than 30 minutes. In some embodiments the one or more post-operative atrial fibrillation episodes have a duration of equal to or greater than 1 hour. In some embodiments the one or more post-operative atrial fibrillation episodes have a duration of equal to or greater than 4 hours.

In some embodiments, the one or more post-operative atrial fibrillation episodes have a duration of equal to or greater than 30 seconds, equal to or greater than 30 minutes, equal or greater than 4 hours, or combinations thereof. In some embodiments, the one or more post-operative atrial fibrillation episodes have a duration equal to or greater than 30 seconds, equal to or greater than 2 minutes, equal to or greater than 5 minutes, or combinations thereof. In some embodiments, the one or more post-operative atrial fibrillation episodes have a duration equal to or greater than 30 seconds, equal to or greater than 1 hour, and equal to or greater than 6 hours, or combinations thereof.

In some embodiments, administering the botulinum toxin serotype A to the subject reduces the occurrence or likelihood of one or more post-operative atrial fibrillation episodes by at least 50% during a period of 30 days from administration, as compared to a subject that does not receive administration of the toxin serotype A while undergoing open-chest CABG surgery, and wherein the one or more atrial fibrillation episodes have a duration of 30 seconds or more.

In some embodiments the one or more post-operative atrial fibrillation episodes have a duration selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours during a period of 30 days from administration. In some embodiments the one or more post-operative atrial fibrillation episodes have a duration of equal to or greater than 30 seconds. In some embodiments the one or more post-operative atrial fibrillation episodes have a duration of equal to or greater than 5 minutes. In some embodiments the one or more post-operative atrial fibrillation episodes have a duration of equal to or greater than 30 minutes. In some embodiments the one or more post-operative atrial fibrillation episodes have a duration of equal to or greater than 1 hour. In some embodiments the one or more post-operative atrial fibrillation episodes have a duration of equal to or greater than 4 hours.

In some embodiments, the one or more post-operative atrial fibrillation episodes have a duration of equal to or greater than 30 seconds, equal to or greater than 30 minutes, equal or greater than 4 hours, or combinations thereof. In some embodiments, the one or more post-operative atrial fibrillation episodes have a duration equal to or greater than 30 seconds, equal to or greater than 2 minutes, equal to or greater than 5 minutes, or combinations thereof. In some embodiments, the one or more post-operative atrial fibrillation episodes have a duration equal to or greater than 30 seconds, equal to or greater than 1 hour, and equal to or greater than 6 hours, or combinations thereof.

In some embodiments, the administering further reduces the occurrence or likelihood of hospital readmission of the subject within 30 days after discharge by at least 30% as compared to a subject that does not receive administration of the botulinum toxin serotype A while undergoing open-chest CABG surgery. In some embodiments, the administering further reduces the occurrence or likelihood of hospital readmission of the subject within 30 days after discharge by at least 40% as compared to a subject that does not receive administration of the botulinum toxin serotype A while undergoing open-chest CABG surgery. In some embodiments, the administering further reduces the occurrence or likelihood of hospital readmission of the subject within 30 days after discharge by at least 50% as compared to a subject that does not receive administration of the botulinum toxin serotype A while undergoing open-chest CABG surgery.

In other aspects, the present disclosure provides a method of reducing the occurrence or likelihood of hospital readmission within 60 days after discharge after open-chest cardiac surgery in a subject in need thereof, comprising (1) selecting a subject in need of open-chest coronary artery bypass grafting (CABG), and (2) administering to five epicardial fat pads of the subject about 125 units of a botulinum toxin serotype A by injection while the subject is undergoing open-chest cardiac surgery, wherein the five epicardial fat pads comprise an aortic fat pad, a superior right-side pulmonary vein fat pad, an anterior right-side pulmonary vein fat pad, a superior left-side pulmonary vein fat pad, and an anterior left-side pulmonary vein fat pad, thereby reducing the occurrence or likelihood of hospital readmission within 60 days after discharge after open-chest CABG surgery. In some embodiments, the method reduces the occurrence or likelihood of hospital readmission within 30 days after discharge.

In one embodiment, the method reduces the occurrence or likelihood of all-cause hospital readmission within 60-day after discharge. In one embodiment, the method reduces the occurrence or likelihood of all-cause hospital readmission within 30-day after discharge. In one embodiment, the method reduces the occurrence or likelihood of cardiac-related hospital readmission. In one embodiment, the method reduces the occurrence or likelihood of cardiac-related hospital readmission within 30 days after discharge.

In some embodiments, the method further comprises selecting a subject that is about 65 years of age or older.

In some embodiments, the method further comprises selecting a subject that is not in need of valve surgery.

In some embodiments, the administering reduces the occurrence or likelihood of hospital readmission of the subject within 30 days after discharge by at least 10% as compared to a subject that does not receive administration of the botulinum toxin serotype A while undergoing open-chest CABG surgery.

In some embodiments, the administering reduces the occurrence or likelihood of hospital readmission of the subject within 30 days after discharge by at least 20% as compared to a subject that does not receive administration of the botulinum toxin serotype A while undergoing open-chest CABG surgery.

In some embodiments, the administering reduces the occurrence or likelihood of hospital readmission of the subject within 30 days after discharge by at least 30% as compared to a subject that does not receive administration of the botulinum toxin serotype A while undergoing open-chest CABG surgery.

In some embodiments, the administering reduces the occurrence or likelihood of hospital readmission of the subject within 30 days after discharge by at least 40% as compared to a subject that does not receive administration of the botulinum toxin serotype A while undergoing open-chest CABG surgery.

In some embodiments, the administering reduces the occurrence or likelihood of hospital readmission of the subject within 30 days after discharge by at least 50% as compared to a subject that does not receive administration of the botulinum toxin serotype A while undergoing open-chest CABG surgery.

In some embodiments, the methods described herein further comprise selecting a subject that has a history of paroxysmal atrial fibrillation or a history of persistent atrial fibrillation.

In some embodiments, the methods described herein further comprising selecting a subject that does not have a history of paroxysmal atrial fibrillation or a history of persistent atrial fibrillation.

In some embodiments, the methods further result in a reduction in length of stay in intensive care unit (ICU) of at least 8 hours for the subject as compared to a subject that does not receive administration of the botulinum toxin serotype A while undergoing open-chest CABG surgery. In some embodiments, the methods further result in a reduction in length of stay in intensive care unit (ICU) by at least 0.5 day for the subject as compared to a subject that does not receive administration of the botulinum toxin serotype A while undergoing open-chest CABG surgery. In some embodiments, the methods further result in a reduction in length of stay in intensive care unit (ICU) of at least 1 day for the subject as compared to a subject that does not receive administration of the botulinum toxin serotype A while undergoing open-chest CABG surgery. In some embodiments, the methods further result in a reduction in length of stay in intensive care unit (ICU) of at least 2 days for the subject as compared to a subject that does not receive administration of the botulinum toxin serotype A while undergoing open-chest CABG surgery. In some embodiments, the methods further result in a reduction in length of stay in intensive care unit (ICU) of at least 3 days for the subject as compared to a subject that does not receive administration of the botulinum toxin serotype A while undergoing open-chest CABG surgery. In some embodiments, the methods further result in a reduction in length of stay in intensive care unit (ICU) of at least 4 days, 5 days, 6 days, 7 days, 8 days, 9 days, or up to 10 days for the subject as compared to a subject that does not receive administration of the botulinum toxin serotype A while undergoing open-chest CABG surgery.

In some embodiments, the methods further result in a reduction in hospital length of stay by at least 0.5 days. In some embodiments, the methods further result in a reduction in hospital length of stay by at least 1 day, 2 days, 3 days, 4 days, 5 days, or up to 10 days for the subject as compared to a subject that does not receive administration of the botulinum toxin serotype A while undergoing open-chest CABG surgery.

In some embodiments, the methods further result in a reduction of anticoagulant usage for the subject during the first 30 days from administration by at least 30% as compared to a subject that does not receive administration of the botulinum toxin serotype A while undergoing open-chest CABG surgery.

In one aspect, disclosed herein is a method of reducing anticoagulant usage after open-chest CABG surgery in a subject in need thereof, comprising selecting a subject in need of open-chest coronary artery bypass grafting (CABG), administering to five epicardial fat pads of the subject about 125 units of a botulinum toxin serotype A by injection while the subject is undergoing open-chest CABG surgery, wherein the five epicardial fat pads comprise an aortic fat pad, a superior right-side pulmonary vein fat pad, an anterior right-side pulmonary vein fat pad, a superior left-side pulmonary vein fat pad, and an anterior left-side pulmonary vein fat pad, wherein the administering reduces anticoagulant usage by at least 20% during a period of 30 days from administration, as compared to a subject that does not receive administration of the toxin serotype A while undergoing open-chest CABG surgery.

In one embodiment, the method further comprises selecting a subject who is not in need of valve surgery.

In some embodiments, the methods further result in a reduction of anticoagulant usage for the subject during the first 30 days from administration by at least 40% as compared to a subject that does not receive administration of the botulinum toxin serotype A while undergoing open-chest CABG surgery.

In some embodiments, the methods increase time to first anticoagulant use by at least 1 day.

In some embodiment, the administering step in the methods reduces the occurrence or likelihood of one or more post-operative atrial fibrillation episodes by at least 40% during a period of 30 days from administration, wherein the one or more atrial fibrillation episodes have a duration equal to or greater than 30 seconds, equal to or greater than 1 hour, equal to or greater than 6 hours, or combinations thereof, as compared to a subject that does not receive administration of the toxin serotype A while undergoing open-chest CABG surgery.

In some embodiments, the administering step further reduces hospital length of stay by at least 1 day. In some embodiments, the administering step further reduces length of stay in intensive care unit (ICU) by at least 0.5 days. In some embodiments, the administering step further reduces the occurrence or likelihood in hospital readmission of the subject within 30 days after discharge by at least 40% as compared to a subject that does not receive administration of the botulinum toxin serotype A while undergoing open-chest CABG surgery. In some embodiments, the administering step further increases the time to first anticoagulant use in 30 days post-surgery at least 30% as compared to a subject that does not receive administration of the botulinum toxin serotype A while undergoing open-chest CABG surgery.

In some embodiments, the subject has received a beta-blocker therapy and is withdrawn from the beta-blocker therapy after, e.g., about 1 day, 2 days, or 3 days after the open-chest CABG surgery and not restarting beta-blocker therapy for, e.g., at least 7 days after the open-chest CABG surgery.

In some embodiments, the administering of the botulinum toxin serotype A to the subject withdrawn from the beta-blocker therapy reduces the occurrence or likelihood of one or more post-operative atrial fibrillation episodes by at least 20% during a period of 30 days from administration, as compared to a subject that does not receive administration of the toxin serotype A while undergoing open-chest CABG surgery, and wherein the one or more atrial fibrillation episodes have a duration of 30 seconds or more. In one embodiment, the one or more atrial fibrillation episodes have a duration of 5 minutes or more. In one embodiment, the one or more atrial fibrillation episodes have a duration of 30 minutes or more. In one embodiment, the one or more atrial fibrillation episodes have a duration of 1 hour or more. In one embodiment, the one or more atrial fibrillation episodes have a duration of 4 hours or more.

In some embodiments, the administering of the botulinum toxin serotype A to the subject withdrawn from the beta-blocker therapy reduces the occurrence or likelihood of one or more post-operative atrial fibrillation episodes by at least 40% during a period of 30 days from administration, as compared to a subject that does not receive administration of the toxin serotype A while undergoing open-chest CABG surgery, and wherein the one or more atrial fibrillation episodes have a duration of 30 seconds or more. In one embodiment, the one or more atrial fibrillation episodes have a duration of 5 minutes or more. In one embodiment, the one or more atrial fibrillation episodes have a duration of 30 minutes or more. In one embodiment, the one or more atrial fibrillation episodes have a duration of 1 hour or more. In one embodiment, the one or more atrial fibrillation episodes have a duration of 4 hours or more.

In some embodiments, the administering of the botulinum toxin serotype A to the subject withdrawn from the beta-blocker therapy reduces the occurrence or likelihood of one or more post-operative atrial fibrillation episodes by at least 50% during a period of 30 days from administration, as compared to a subject that does not receive administration of the toxin serotype A while undergoing open-chest CABG surgery, and wherein the one or more atrial fibrillation episodes have a duration of 30 seconds or more. In one embodiment, the one or more atrial fibrillation episodes have a duration of 5 minutes or more. In one embodiment, the one or more atrial fibrillation episodes have a duration of 30 minutes or more. In one embodiment, the one or more atrial fibrillation episodes have a duration of 1 hour or more. In one embodiment, the one or more atrial fibrillation episodes have a duration of 4 hours or more.

In certain aspects, the present disclosure provides a method of reducing anticoagulant usage after open-chest CABG surgery in a subject in need thereof, comprising: (1) selecting a subject in need of open-chest coronary artery bypass grafting (CABG), (2) administering to five epicardial fat pads of the subject about 125 units of a botulinum toxin serotype A by injection while the subject is undergoing open-chest CABG surgery, wherein the five epicardial fat pads comprise an aortic fat pad, a superior right-side pulmonary vein fat pad, an anterior right-side pulmonary vein fat pad, a superior left-side pulmonary vein fat pad, and an anterior left-side pulmonary vein fat pad, wherein the administering reduces anticoagulant usage by at least 10% during a period of 30 days from administration, as compared to a subject that does not receive administration of the toxin serotype A while undergoing open-chest CABG surgery. In one embodiment, the administering reduces anticoagulant usage by at least 20% during a period of 30 days from administration. In one embodiment, the administering reduces anticoagulant usage by at least 30% during a period of 30 days from administration. In one embodiment, the administering reduces anticoagulant usage by at least 40% during a period of 30 days from administration. In one embodiment, the administering reduces anticoagulant usage by at least 50% during a period of 30 days from administration.

In some embodiments, reducing anticoagulant usage comprises delaying the time it takes to first anticoagulant use, reducing the amount of anticoagulant used, or both, during a period, e.g., a period of 30 days, after open-chest CABG surgery.

In some embodiments, the administering reduces anticoagulant usage for the subject by at least 30% during a period of 30 days from administration, as compared to a subject that does not receive administration of the botulinum toxin serotype A while undergoing open-chest CABG surgery.

In some embodiments, the administering reduces anticoagulant usage for the subject by at least 40% during a period of 30 days from administration, as compared to a subject that does not receive administration of the botulinum toxin serotype A while undergoing open-chest CABG surgery.

In some embodiments, the administering comprises administering about 10 units to about 40 units of the botulinum toxin serotype A to each of the five epicardial fat pads, for a total dose of about 125 units.

In some embodiments, the administering comprises administering about 20 units to about 30 units of the botulinum toxin serotype A to each of the five epicardial fat pads, for a total dose of about 125 units.

In some embodiments, the administering comprises administering about 25 units of the botulinum toxin serotype A to each of the five epicardial fat pads, for a total dose of about 125 units.

In some embodiments, the administering comprises injecting 1 mL of a botulinum toxin serotype A solution having a concentration of 25 units/mL into each of the five epicardial fat pads.

In some embodiments, the administering comprises injecting 0.5 mL of a botulinum toxin serotype A solution having a concentration of 50 units/mL into each of the five epicardial fat pads.

In some embodiments, the botulinum toxin serotype A is animal protein free.

In some embodiments, the botulinum toxin serotype A is selected from daxibotulinumtoxinA, nivobotulinumtoxinA, and gemibotulinumtoxinA.

In some embodiments, the botulinum toxin serotype A is gemibotulinumtoxinA.

In some embodiments, gemibotulinumtoxinA is provided in a lyophilized formulation comprising trehalose, poloxamer P188, L-methionine and histidine.

In some embodiments, gemibotulinumtoxinA is provided in a solution. In one embodiment, gemibotulinumtoxinA is provided in a solution comprising trehalose, poloxamer P188, L-methionine and histidine.

In some embodiments, the botulinum toxin serotype A is a botulinum toxin serotype A complex.

In some embodiments, the botulinum toxin serotype A is a pure toxin. In some embodiments, the botulinum toxin serotype A is incobotulinumtoxinA. In some embodiments, the botulinum toxin serotype A is daxibotulinumtoxinA.

In some embodiments, the botulinum toxin serotype A is not co-administered with a second agent selected from the group consisting of a drug that interferes with neuromuscular transmission, an aminoglycoside, an anticholinergic drug, or a muscle relaxant.

In some embodiments, the administering comprises injecting the botulinum toxin serotype A at 1-5 locations per fat pad.

In some embodiments, the administering comprises injecting the botulinum toxin serotype A at 1, 2 or 3 locations per fat pad.

In some embodiments, the time for administering is about 5-30 seconds per injection, or 5-150 seconds per fat pad. In some embodiments, the time for administering is about 10-15 seconds per injection, or 10-45 seconds per fat pad.

In some embodiments, injections are made at a depth of about 1-2 mm.

In some embodiments, injections are made at an oblique angle.

In some embodiments, the administering comprises injecting the botulinum toxin serotype A at one location of each of the superior and anterior right-side pulmonary vein fat pads and the superior and anterior left-side pulmonary vein fat pads.

In some embodiments, the botulinum toxin serotype A is first administered to the aortic fat pad.

In some embodiments, the methods do not comprise electrically stimulating the epicardial fat pads to locate an administration site on the epicardial fat pads.

In some embodiments, the administering comprises administering the botulinum toxin serotype A to the epicardial fat pads in a sequential order of the aortic fat pad, the superior and anterior right-side pulmonary vein fat pads, and the superior and anterior two left-side pulmonary vein fat pads.

In some embodiments, the administering comprises administering the effective amount of botulinum toxin serotype A to the epicardial fat pads in a sequential order of the aortic fat pad, the superior and anterior left-side pulmonary vein fat pads, and the superior and anterior two right-side pulmonary vein fat pads.

In some embodiments, administering in the sequential order reduces time for administering the effective amount to the epicardial fat pads relative to administering the effective amount in an order other than the sequential order.

In some embodiments, the administering reduces risk of leakage and tissue trauma.

In some embodiments, the administering is performed after pericardial sac is dissected and before the primary surgical procedure.

In some embodiments, the administering comprises administering (i) before cardiopulmonary bypass is initiated, (ii) while bypass cannulas are being placed on the subject; (iii) while the subject is on cardiopulmonary bypass, (iv) after cross-clamping has been performed, and/or (v) after cardioplegia has been instituted.

In some embodiments, the administering comprises administering the botulinum toxin serotype A into the aortic fat pad while the bypass cannulas are being inserted; and then administering the botulinum toxin serotype A into the pulmonary vein fat pads while the subject is on cardiopulmonary bypass.

In some embodiments, the administering comprises administering botulinum toxin serotype A into the aortic fat pad, the superior and anterior right-side pulmonary vein fat pads, and the superior and anterior left-side pulmonary vein fat pads while the subject is on cardiopulmonary bypass.

In some embodiments, the administering comprises administering botulinum toxin serotype A into the aortic fat pad before cardiopulmonary bypass is initiated and into the pulmonary vein fat pads while the subject is on cardiopulmonary bypass.

In some embodiments, the subject is undergoing an off-pump bypass and wherein the botulinum toxin serotype A is administered when the subject is undergoing off-pump bypass surgery.

In some embodiments, the administering comprises injecting botulinum toxin serotype A via a syringe to the aortic fat pad wherein the syringe has a needle bent at an angle ranging from 10 to 90 degrees.

In some embodiments, upon insertion of the needle into the aortic fat pad, the syringe is drawn back to ensure the needle is not inside the aorta.

In some embodiments, the administering comprises injecting botulinum toxin serotype A via a syringe to each of the superior and anterior right-side pulmonary vein fat pads wherein the syringe has a needle bend at an angle ranging from 10 to 90 degrees.

In some embodiments, upon insertion of the needle into one or both of the superior and anterior right-side pulmonary vein fat pads, the syringe is drawn back to ensure the needle is not inside the pulmonary vein or atria.

In some embodiments, the injecting via a syringe to each of the superior and anterior right-side pulmonary vein fat pads wherein the syringe has a needle bend at an angle ranging from 10 to 90 degrees allows for quicker deposits of the botulinum toxin serotype A and reduces the need for manipulation of the heart.

In some embodiments, the administering comprises injecting botulinum toxin serotype A via a syringe to each of the superior and anterior left-side pulmonary vein fat pads, wherein the syringe has a needle, and wherein the needle of the syringe is bent at an angle ranging from 10 to 90 degrees.

In some embodiments, upon insertion of the needle into one or both of the superior and anterior left-side pulmonary vein fat pads, the syringe is drawn back to ensure the needle is not inside the pulmonary vein or atria.

In some embodiments, needle gauge ranges from 25 to 30.

In some embodiments, the needle length of the syringe ranges from 0.5 to 3.5 inch.

In some embodiments, the needle is a spinal needle. In some embodiments, the spinal needle is used to administer the botulinum toxin to the superior and anterior left-sided pulmonary vein fat pads.

In some embodiments, forceps are used to guide the needle.

### BRIEF DESCRIPTION OF THE DRAWINGS

FIG. 1 shows an anterior view of the heart, which illustrates the aortic fat pad and approximate potential injection sites.

FIG. 2 shows a posterior view of the heart, which illustrates the two left-side pulmonary vein fat pads and the two right-side pulmonary vein fat pads. In the present disclosure, the terms left-side pulmonary vein fat pads are also referred to as the left pulmonary vein fat pads, left-side or left pulmonary vein epicardial fat pads, or left-side or left pulmonary vein fat pads; and the right-side pulmonary vein fat pads are also referred to as the right pulmonary vein fat pads, right-side or right pulmonary vein epicardial fat pads, or right-side or right pulmonary vein fat pads. The left pulmonary vein fat pads include the superior and anterior left pulmonary vein fat pads. The right pulmonary vein fat pads include the superior and anterior right pulmonary vein fat pads. The anterior pulmonary vein fat pads are also referred to as the inferior pulmonary vein fat pads.

FIG. 3 shows proportion of participants with at least one continuous atrial fibrillation (AF) episode among patients who received coronary artery bypass graft (CABG) surgery without valve surgery. The patients received placebo, 125U toxin or 250U toxin treatment during the surgery procedure.

FIG. 4 shows proportion of participants with at least one continuous AF episode among patients who were 65 years of age or older. The patients received placebo, 125U toxin or 250U toxin treatment during the surgery procedure.

FIG. 5 shows proportion of participants with at least one continuous AF episode among patients 65 years of age or older and who received coronary artery bypass graft (CABG) surgery without valve surgery. The patients received placebo, 125U toxin or 250U toxin treatment during the surgery procedure.

FIG. 6 is a Kaplan-Meier plot showing probability of AF-free over time during the first 30 days post-surgery among patients 65 years of age or older and who received coronary artery bypass graft (CABG) surgery without valve surgery. The patients received placebo, 125U toxin or 250U toxin treatment during the surgery procedure.

FIG. 7 shows the number of patients in the modified intention-to-treat (mITT) population re-hospitalized due to any causes during the first 30 days post-discharge.

FIG. 8 is a Kaplan-Meier plot showing probability of Rehospitalization-free over time during the first 60 days post-surgery in the modified intention-to-treat (mITT) population. The patients received placebo, 125U toxin or 250U toxin treatment during the surgery procedure. Participants with no hospitalization during the first 60 days post-surgery were censored at day 60, or at their respective study exit day if they exited the study before day 60.

FIG. 9 shows the number and relative percentage of patients in the modified intention-to-treat (mITT) population re-hospitalized due to cardiovascular related causes during the first 30 days post-discharge, where CV is abbreviated for “cardiovascular”, Afib is abbreviated for “Atrial Fibrillation” and Aflu is abbreviated for “atrial flutter”, which is another form of atrial fibrillation.

FIG. 10 is a Kaplan-Meier plot showing probability of Intervention-free over time during the first 30 days post-surgery in the modified intention-to-treat (mITT) population. The Intervention in FIG. 10 is specific to anticoagulant usage. The patients received placebo, 125 U toxin or 250 U toxin treatment during the surgery procedure. Participants with no intervention during the first 30 days post-surgery were censored at day 30, or at their respective study exit day if they exited the study before day 30.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

### I. Definitions

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

As used herein, the words or terms set forth below have the following definitions:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

“Animal” means a mammal (such as a human), bird, reptile, fish, insect, spider or other animal species. “Animal” excludes microorganisms, such as bacteria. An “animal protein free” pharmaceutical composition can include a botulinum neurotoxin. For example, an “animal protein free” pharmaceutical composition means a pharmaceutical composition which is either substantially free or essentially free or entirely free of a serum derived albumin, gelatin and other animal derived proteins, such as immunoglobulins. An example of an animal protein free pharmaceutical composition is a pharmaceutical composition which comprises, or which consists of a botulinum toxin (as the active ingredient) and a suitable polysaccharide as a stabilizer or excipient.

“Before the primary surgery or surgical procedure” (such as coronary artery bypass grafting or valve repair/replacement) refers to the time before the primary surgical procedure and can include: (i) before cardiopulmonary bypass is initiated (i.e. prior to insertion of bypass cannulas), (ii) while bypass cannulas are being placed on the patient, (iii) while the patient is on cardiopulmonary bypass, (iv) after cross-clamping has been performed, and (v) after cardioplegia has been instituted.

“Biological activity” describes the beneficial or adverse effects of a drug on living matter. When a drug is a complex chemical mixture, this activity is exerted by the substance's active ingredient but can be modified by the other constituents. Biological activity can be assessed as potency or as toxicity by an in vivo LD₅₀ or ED₅₀ assay, or through an in vitro assay such as, for example, cell-based potency assays as described in U.S. 2010/0203559 and U.S. 2010/0233802.

“Botulinum toxin” means a neurotoxin produced by Clostridium botulinum, as well as a botulinum toxin (or the light chain or the heavy chain thereof) made recombinantly by a non-Clostridial species. The phrase “botulinum toxin”, as used herein, encompasses Botulinum toxin serotype A (BoNT/A), Botulinum toxin serotype B (BoNT/B), Botulinum toxin serotype C (BoNT/C), Botulinum toxin serotype D (BoNT/D), Botulinum toxin serotype E (BoNT/E), Botulinum toxin serotype F (BoNT/F), Botulinum toxin serotype G (BoNT/G), Botulinum toxin serotype H (BoNT/H), Botulinum toxin serotype X (BoNT/X), and mosaic Botulinum toxins and/or subtypes and variants thereof. In some embodiments, the clostridial derivative is a botulinum toxin, which is selected from the group consisting of botulinum toxin types A, B, C₁, D, E, F and G and mosaics (CD, DC, FA) and non-clostridial BoNT-like encoding sequences (BoNT/X, BoNT/Wo, BoNT/En (eBoNT/J), Cp1, PMP1). “Botulinum toxin”, as disclosed herein, includes, without limitation, naturally occurring botulinum toxins, fragments, or chimeras thereof; non-naturally occurring botulinum toxins, such as recombinant, modified botulinum toxins, fragments, or chimeras thereof. Further, “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins. Throughout this disclosure, “botulinum toxin type A” and “botulinum toxin serotype A” are used interchangeably.

“Cardiopulmonary bypass” or “cardiac bypass” is a procedure in which a heart-lung bypass machine takes over the function of the heart and lung during a patient's surgery, maintaining the circulation of blood and the oxygen content of the patient's body. Cardiopulmonary bypass mechanically circulates and oxygenates blood for the body while bypassing the heart and lungs. Cardiopulmonary Bypass (CPB) is typically achieved in the following sequence: 1) cannulas are placed (one in the right atrium [right atrial appendage] and one into the aorta), 2) blood is diverted from the patient's heart to be oxygenized artificially, 3) the heart remains beating, while the blood is artificially oxygenated (deoxygenated blood is pulled from the right atrium cannula, then oxygenated, then delivered to the aorta cannula). If the heart is to be arrested, the aorta is then cross-clamped distal to the cannula placement to prevent potential backflow of blood and cardioplegia (i. e., cardiac arrest) is induced, in some cases with cold potassium and/or other ion solutions.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/C₁, a BoNT/D, a BoNT/E, a BoNT/F, a BoNT/G, a BoNT/H, a BoNT/X, a mosaic Botulinum toxin and/or subtypes and variants thereof, a Tetanus toxin (TeNT), a Baratii toxin (BaNT), and a Butyricum toxin (BuNT). Non-limiting examples of Clostridial toxins include a Botulinum toxin like BoNT/A, a BoNT/B, a BoNT/C₁, a BoNT/CD, BoNT/D, a BoNT/DC a BoNT/E, a BoNT/F, a BoNT/FA, a BoNT/G, a BoNT/X, an Enterococcus faecium toxin (BoNT/En also called eBoNT/J), a Weissella oryzae toxin (BoNT/Wo), a Chryseobacterium piperi toxin (Cp1), a Paraclostridium bifermentans toxin (PMP1), a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

“Coronary artery bypass grafting” (CABG), also called heart bypass surgery, is a medical procedure to improve blood flow to the heart. It may be needed when the arteries supplying blood to the heart, called coronary arteries, are narrowed or blocked. CABG uses healthy blood vessels from another part of the body and connects them to blood vessels above and below the blocked artery. This creates a new route for blood to flow that bypasses the narrowed or blocked coronary arteries. The blood vessels are usually arteries from the arm or chest, or veins from the legs.

“Effective amount” as applied to the biologically active ingredient means that amount of the ingredient which is generally sufficient to effect a desired change in the subject. For example, where the desired effect is a reduction in pain or another symptom of a disorder, an effective amount of the ingredient is that amount which causes at least a substantial reduction of the pain or symptom, and without resulting in significant toxicity.

“Elective coronary artery bypass grafting”, or “elective CABG,” as used herein, refer to a CABG surgery wherein a subject's cardiac function has been stable in the days or weeks before the surgery, “emergent CABG” is performed on patients who are already in hospital and/or need surgery within 1-5 days post diagnosis (of the need for cardiac surgery), and “emergency CABG” represents surgeries that must be performed in emergency situation, perhaps <24 hours post diagnosis, when a patient is not responding to other treatment.

“Isolated coronary artery bypass grafting”, or “isolated CABG,” as used herein, refer to surgery in which only a CABG surgery is performed on a subject without, e.g., cardiac valve surgery. In many embodiments, the term “isolated CABG” may be used interchangeably with the term “absence of valve surgery”.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient's skin.

“Modified botulinum toxin” means a botulinum toxin that has had at least one of its amino acids deleted, modified, or replaced, as compared to a native botulinum toxin. Additionally, the modified botulinum toxin can be a recombinantly produced neurotoxin, or a derivative or fragment of a recombinantly made neurotoxin. A modified botulinum toxin retains at least one biological activity of the native botulinum toxin, such as, the ability to bind to a botulinum toxin receptor, or the ability to inhibit neurotransmitter release from a neuron. One example of a modified botulinum toxin is a botulinum toxin that has a light chain from one botulinum toxin serotype (such as serotype A), and a heavy chain from a different botulinum toxin serotype (such as serotype B). Another example of a modified botulinum toxin is a botulinum toxin coupled to a neurotransmitter, such as substance P.

“Mutation” means a structural modification of a naturally occurring protein or nucleic acid sequence. For example, in the case of nucleic acid mutations, a mutation can be a deletion, addition or substitution of one or more nucleotides in the DNA sequence. In the case of a protein sequence mutation, the mutation can be a deletion, addition or substitution of one or more amino acids in a protein sequence. For example, a specific amino acid comprising a protein sequence can be substituted for another amino acid, for example, an amino acid selected from a group which includes the amino acids alanine, asparagine, cysteine, aspartic acid, glutamic acid, phenylalanine, glycine, histidine, isoleucine, lysine, leucine, methionine, proline, glutamine, arginine, serine, threonine, valine, tryptophan, tyrosine or any other natural or non-naturally occurring amino acid or chemically modified amino acids. Mutations to a protein sequence can be the result of mutations to DNA sequences that when transcribed, and the resulting mRNA translated, produce the mutated protein sequence. Mutations to a protein sequence can also be created by fusing a peptide sequence containing the desired mutation to a desired protein sequence.

“On-pump surgery” refers to a cardiac surgery conducted with cardiopulmonary bypass.

“Off-bypass” or “off-pump bypass” refers to cardiac surgeries which are conducted without cardiopulmonary bypass.

“Open-heart surgery” refers to any surgery done on the heart muscle, valves, arteries, or the aorta and other large arteries connected to the heart. Open-heart surgery requires opening the chest wall to make the heart accessible. The term “open-heart surgery” is used interchangeably herein with “open-chest surgery” or “open-chest cardiac surgery”. Open-heart surgeries include but are not limited to Coronary artery bypass grafting (CABG), heart valve repair or replacement, insertion of a pacemaker or an implantable cardioverter defibrillator (ICD), maze surgery, aneurysm repair, and heart transplant.

“Patient” means a human subject receiving medical or veterinary care. In many embodiments, the term “patient” may be used interchangeably with the term “subject.”

“Paroxysmal atrial fibrillation”, as used herein, refers to an atrial fibrillation episode that is intermittent and may terminate spontaneously or within 7 days from onset. “Persistent atrial fibrillation”, as used herein, refers to an atrial fibrillation episode or episodes that are experienced over more than 7 days.

“Pharmaceutical composition” means a composition comprising an active pharmaceutical ingredient, such as, for example, a clostridial toxin active ingredient such as a botulinum toxin, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Post-operative atrial fibrillation” or “POAF” refers to atrial fibrillation that occurs after cardiac surgery. POAF can occur within a time interval post-surgery, such as within 30 days post-surgery, within 60 days post-surgery, within 90 days post-surgery, within 180 days post-surgery, within one-year post-surgery, within three years post-surgery, or after 3 years post-surgery.

“Preventing”, “preventing”, or “prevention” refers to a method of preventing the onset of a disease and/or its attendant symptoms or barring a subject from acquiring a disease. As used herein, “prevent”, “preventing” and “prevention” also include: (i) delaying the onset of a disease and/or its attendant symptoms, (ii) reducing a subject's risk or likelihood of acquiring or developing a disease or disorder, (iii) reducing the prevalence, occurrence, incidence, frequency of the disease and/or its attendant symptoms, and (iv) reducing the severity of the disease and/or its attendant symptoms.

“Subject” as used herein refers to a mammal (e.g., rat, mouse, cat, dog, cow, sheep, horse, goat, rabbit), preferably a human, for example, in need of medical or veterinary care. In some embodiments, the subject needs coronary artery bypass grafting (CABG). In some embodiments, the subject needs elective and/or emergent CABG. In some embodiments, the subject does not need cardiac valve surgery. In many embodiments, the term “subject” may be used interchangeably with the term “patient.” The expression “a subject in need thereof” refers to a human that needs open-chest cardiac surgery, e.g., open-chest CABG surgery, and is at risk of having post-operative atrial fibrillation.

“Treating” means to alleviate or eliminate at least one symptom of a condition or disorder, such as, for example, spasticity, depression, pain (such as, for example, headache pain) or the like, either temporarily or permanently.

“Unit” or “U” refers to the LD₅₀ dose or the dose determined by a cell-based potency assay (CBPA). The LD₅₀ dose is defined as the amount of a Clostridial toxin active ingredient, Clostridial toxin complex or modified Clostridial toxin that killed 50% of the mice injected with the Clostridial toxin, Clostridial toxin complex or modified Clostridial toxin. The CBPA dose is determined as described in U.S. Pat. Nos. 8,618,261; 8,198,034; 9,249,216; 10,703,806; 11,261,240 and 11,332,518; the assay details of which are incorporated by reference herein.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. Methods of Treatment

In certain aspects, the present disclosure provides methods for preventing post-operative atrial fibrillation (POAF) in a subject undergoing open-chest CABG surgery by administering a botulinum toxin serotype A, a paralysis-causing neurotoxin, to five epicardial fat pads of the subject during surgery. In some embodiments, the botulinum toxin serotype A is administered in a dosage of about 125 units. In some embodiments, the botulinum toxin serotype A is administered in a dosage of about 250 units.

In some other aspects, the present disclosure provides methods for preventing hospital readmission after discharge after open-chest CABG surgery in a subject in need thereof, by administering a dosage of a botulinum toxin serotype A to five epicardial fat pads of the subject during surgery. In some embodiments, the dosage is about 125 units. In some embodiments, the dosage is about 250 units.

In some other aspects, the present disclosure provides methods for reducing anticoagulant usage after open-chest CABG surgery in a subject in need thereof, by administering a dosage of a botulinum toxin serotype A to five epicardial fat pads of the subject during surgery. In some embodiments, the dosage is about 125 units. In some embodiments, the dosage is about 250 units.

Specific regions of the heart contain dense clusters of nerve tissue which innervate myocardial tissues and help modulate heart rate and rhythm. These nerve clusters are often referred to as ganglionic plexi. Ganglionic plexi are loci of the cardiac autonomic system and thus contribute to control of the nerve activity that reaches the heart. The majority of these cardiac ganglionic plexi are embedded in epicardial fat, often referred to as epicardial fat pads, and these discrete epicardial fat pads are located at key junctions in the heart (most notably, the four regions adjacent to or at the four pulmonary vein/atrial junctions and one region near the base of the aorta).

It is hypothesized that a surge in neural activity following an open-chest cardiac surgical procedure may contribute to the initiation and propagation of atrial fibrillation. Alterations in autonomic neural tone, parasympathetic or sympathetic, have known, mechanistic, pro-arrhythmic influences on the initiation and continuation of atrial fibrillation, including in the electrophysiological characteristics of the underlying cardiac tissue and myocardium.

Peri- and post-operative inflammatory processes (such as elevations in interleukin-6 and/or c-reactive protein) are hypothesized to influence the cardiac ganglionic plexi and their neural activities. By modulating the ganglionic plexi in those epicardial fat pads described herein, this neural activity could be suppressed, and this neural suppression could reduce the risk of patients developing atrial fibrillation following cardiac surgery, often referred to as post-operative atrial fibrillation (POAF).

The methods described in the instant disclosure are based on the surprising finding that, administration of 125 units of a botulinum toxin serotype A to five epicardial fat pads of certain subpopulations of subjects, e.g., subjects undergoing CABG, reduces the occurrence or likelihood of post-operative atrial fibrillation, reduces the occurrence or likelihood of hospitalization readmission (i.e., rehospitalization) and reduces anticoagulant usage compared to subjects that do not receive administration of the botulinum toxin serotype A (i.e., receiving placebo). Therefore, according to certain aspects, the present disclosure provides methods for preventing post-operative atrial fibrillation (POAF) in a subject in need thereof, comprising (1) selecting a subject in need of open-chest coronary artery bypass grafting (CABG) surgery, and (2) administering to five epicardial fat pads of the subject about 125 units of a botulinum toxin serotype A while the subject is undergoing open-chest CABG surgery, wherein the five epicardial fat pads comprise an aortic fat pad, a superior right-side pulmonary vein fat pad, an anterior right-side pulmonary vein fat pad, a superior left-side pulmonary vein fat pad, and an anterior left-side pulmonary vein fat pad, thereby preventing post-operative atrial fibrillation in the subject. In some embodiments, the method further comprises selecting a subject in need of elective CABG. In some embodiments, the method further comprises selecting a subject in need of emergent CABG.

In some embodiments, the method further comprises selecting a subject that is about 65 years of age or older. In some embodiments, the subject is at least 65 years old, 70 years old, 75 years old, 80 years old, 85 years old, or 90 years old. In some embodiments, the method further comprises selecting a subject that is about 60 years of age or older.

In some embodiments, the methods further comprise selecting a subject that is not in need of valve surgery (e.g., an isolated CABG subject).

In some embodiments, the methods further comprise selecting a subject that has a history or paroxysmal atrial fibrillation or a history of persistent atrial fibrillation. In some other embodiments, the methods further comprise selecting a subject that does not have a history or paroxysmal atrial fibrillation or a history of persistent atrial fibrillation.

In some embodiments, the methods further comprise selecting a subject that has received a beta-blocker therapy and is to be withdrawn from the beta-blocker therapy after the open-chest CABG surgery.

In some embodiments, the methods comprise not selecting a subject that is about 55 years of age or younger. In some embodiments, the methods comprise not selecting a subject that has used botulinum toxin type A within 6 months prior to the open-chest CABG surgery. In some embodiments, the methods comprise not selecting a subject that is on antiarrhythmic drug therapy. In some embodiments, the methods comprise not selecting a subject that is sensitive to botulinum toxin type A. In some embodiments, the methods comprise not selecting a subject that has been administered a drug that interferes with neuromuscular transmission, an aminoglycoside, an anticholinergic drug, or a muscle relaxant prior to the open-chest CABG surgery.

As used herein, “during a period of 30 days from administration” refers to the 30-day period after administration of the botulinum toxin and the surgery.

The methods of the disclosure reduce in a subject in need of open-chest CABG the occurrence or likelihood of at least one episode of post-operative atrial fibrillation (AF) of equal to or greater than about 30 seconds in the first 30 days after surgery relative to a method wherein a subject receives a placebo rather than the botulinum toxin. The present treatment will also reduce in the subject the measures of AF burden for a time interval after surgery, including the total percentage of time spent in AF, time to occurrence of the first AF event after surgery, and occurrence of at least one or more POAF episodes having a duration ≥2 minutes, ≥5 minutes, ≥6 minutes, ≥30 minutes, ≥1 hour, ≥4 hours, ≥6 hours, ≥12 hours, and ≥24 hours. The time interval after surgery can be within 30 days, 60 days, 90 days, 180 days, 1 year, 3 years or longer than 3 years.

In some embodiments, the subject does not exhibit at least one continuous post-operative atrial fibrillation episode having a duration equal to or greater than 24 hours, 12 hours, 6 hours, 4 hours, 1 hour, 30 minutes, 6 minutes, 5 minutes, 2 minutes, or 30 seconds during a period of 30 days from administration (i.e., within 30 days after the administration of the botulinum toxin).

In some embodiments, the subject does not exhibit at least one continuous post-operative atrial fibrillation episode having a duration greater than 4 hours during a period of 30 days from administration (i.e., within 30 days after the administration of the botulinum toxin serotype A). In some embodiments, the subject does not exhibit at least one continuous post-operative atrial fibrillation episode having a duration greater than 1 hour during a period of 30 days from administration (i.e., within 30 days after the administration of the botulinum toxin serotype A). In some embodiments, the subject does not exhibit at least one continuous post-operative atrial fibrillation episode having a duration greater than 30 minutes during a period of 30 days from administration (i.e., within 30 days after the administration of the botulinum toxin serotype A). In some embodiments, the subject does not exhibit at least one continuous post-operative atrial fibrillation episode having a duration greater than 5 minutes during a period of 30 days from administration (i.e., within 30 days after administration of the botulinum toxin). In some embodiments, the subject does not exhibit at least one continuous post-operative atrial fibrillation episode having a duration greater than 30 seconds during a period of 30 days from administration (i.e., within 30 days after the administration of the botulinum toxin serotype A).

In some embodiments, administering the botulinum toxin serotype A reduces the occurrence or likelihood of post-operative atrial fibrillation episodes in the subject as compared to a subject that does not receive administration of the botulinum toxin serotype A while undergoing open-chest CABG surgery.

In some embodiments, administering the botulinum toxin serotype A to the subject reduces occurrence or likelihood of one or more post-operative atrial fibrillation episodes having a duration equal to or greater than 30 seconds by at least 20%, during a period of 30 days from administration, as compared to a subject that does not receive administration of the toxin serotype A while undergoing open-chest CABG surgery. In some embodiments, administering the botulinum toxin serotype A to the subject reduces the likelihood or occurrence of one or more post-operative atrial fibrillation episodes having a duration equal to or greater than 30 seconds by at least 30%, during a period of 30 days from administration, as compared to a subject that does not receive administration of the toxin serotype A while undergoing open-chest CABG surgery. In some embodiments, administering the botulinum toxin serotype A to the subject reduces the likelihood or occurrence of one or more post-operative atrial fibrillation episodes having a duration equal to or greater than 30 seconds by at least 40%, during a period of 30 days from administration, as compared to a subject that does not receive administration of the toxin serotype A while undergoing open-chest CABG surgery. In some embodiments, administering the botulinum toxin serotype A to the subject reduces the likelihood or occurrence of one or more post-operative atrial fibrillation episodes having a duration equal to or greater than 30 seconds by at least 50%, during a period of 30 days from administration, as compared to a subject that does not receive administration of the toxin serotype A while undergoing open-chest CABG surgery.

In some embodiments, the one or more post-operative atrial fibrillation episodes have a duration equal to or greater than 30 seconds is selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours during a period of 30 days from administration.

In some embodiments, the one or more post-operative atrial fibrillation episodes have a duration equal to or greater than 5 minutes. In some embodiments, the one or more post-operative atrial fibrillation episodes have a duration equal to or greater than 30 minutes. In some embodiments, the one or more post-operative atrial fibrillation episodes have a duration equal to or greater than 1 hour. In some embodiments, the one or more post-operative atrial fibrillation episodes have a duration equal to or greater than 4 hours.

In some embodiments, administering the botulinum toxin serotype A to the subject reduces the occurrence or likelihood of one or more post-operative atrial fibrillation episodes by at least 30% during a period of 30 days from administration, as compared to a subject that does not receive administration of the toxin serotype A while undergoing open-chest CABG surgery, and wherein the one or more atrial fibrillation episodes have a duration equal to or greater than 30 seconds, equal to or greater than 30 minutes, equal to or greater than 4 hours, or combinations thereof. In some embodiments, the one or more post-operative atrial fibrillation episodes have a duration equal to or greater than 30 seconds, equal to or greater than 2 minutes, equal to or greater than 5 minutes, or combinations thereof. In some embodiments, the one or more post-operative atrial fibrillation episodes have a duration equal to or greater than 30 seconds, equal to or greater than 1 hour, and equal or greater than 6 hours, or combinations thereof. In some embodiments, the administering reduces the occurrence or likelihood of one or more post-operative atrial fibrillation episodes by at least 40%. In some embodiments, the administering reduces the occurrence or likelihood of one or more post-operative atrial fibrillation episodes by at least 50%.

In certain aspects, the present disclosure also provides methods for reducing the occurrence or likelihood of hospital readmission (i.e., rehospitalization) after discharge after open-chest CABG surgery in a subject in need thereof, comprising (1) selecting a subject in need of elective coronary artery bypass grafting (CABG), and (2) administering to five epicardial fat pads of the subject 125 units or 250 units of a botulinum toxin serotype A while the subject is undergoing open-chest CABG surgery, thereby reducing the occurrence of hospital readmission after discharge after open-chest CABG surgery.

In some embodiments, administering the botulinum toxin serotype A to the subject prevents hospital readmission due to all causes within 60 days after discharge after open-chest CABG surgery. In some embodiments, administering the botulinum toxin serotype A to the subject prevents hospital readmission due to all causes within 30 days after discharge after open-chest CABG surgery.

In some embodiments, administering the botulinum toxin serotype A to the subject prevents cardiac-related hospital readmission within 60 days after discharge after open-chest CABG surgery. In some embodiments, administering the botulinum toxin serotype A to the subject prevents cardiac related hospital readmission within 30 days after discharge after open-chest CABG surgery.

In some embodiments, the method further comprises selecting a subject that is about 65 years of age or older. In some embodiments, the subject is at least 65 years old, 70 years old, 75 years old, 80 years old, 85 years old, or 90 years old. In some embodiments, the method further comprises selecting a subject that is about 60 years of age or older.

In some embodiments, the methods further comprise selecting a subject that is not in need of valve surgery (e.g., an isolated CABG subject).

In some embodiments, the methods further comprising selecting a subject that has received a beta-blocker therapy and is to be withdrawn from the beta-blocker therapy after the open-chest CABG surgery.

In some embodiments, the subject is not readmitted to the hospital within 10 days, 20 days, 30 days, 45 days, 60 days, 2 months, 4 months, 6 months, 8 months, 10 months or 12 months after discharge after open-chest CABG surgery. In some embodiments, the subject is not rehospitalized within 30 days after discharge after open-chest CABG surgery.

In some embodiments, the administering of the botulinum toxin serotype A reduces the occurrence or likelihood of hospital readmission within 30 days after discharge of the subject by at least about 10%, 20%, 30%, 40%, or 50% as compared to a subject that does not receive administration of the botulinum toxin serotype A while undergoing open-chest CABG surgery.

In some embodiments, the administering of the botulinum toxin serotype A reduces the occurrence or likelihood of hospital readmission of the subject by at least 10% during a period of 30 days from discharge, as compared to a subject that does not receive administration of the toxin serotype A while undergoing open-chest CABG surgery. In some embodiments, the administering of the botulinum toxin serotype A reduces the likelihood or occurrence of hospital readmission of the subject by at least 20% during a period of 30 days from discharge, as compared to a subject that does not receive administration of the toxin serotype A while undergoing open-chest CABG surgery. In some embodiments, the administering of the botulinum toxin serotype A reduces the likelihood or occurrence of hospital readmission of the subject by at least 30% during a period of 30 days from discharge, as compared to a subject that does not receive administration of the toxin serotype A while undergoing open-chest CABG surgery. In some embodiments, the administering of the botulinum toxin serotype A reduces the likelihood or occurrence of hospital readmission of the subject by at least 40% during a period of 30 days from discharge, as compared to a subject that does not receive administration of the toxin serotype A while undergoing open-chest CABG surgery. In some embodiments, the administering of the botulinum toxin serotype A reduces the likelihood or occurrence of hospital readmission of the subject by at least 50% during a period of 30 days from discharge, as compared to a subject that does not receive administration of the toxin serotype A while undergoing open-chest CABG surgery.

In some embodiments, the administering of the botulinum toxin serotype A further reduces the occurrence or likelihood of one or more post-operative atrial fibrillation episodes by at least 30% during a period of 30 days from administration, as compared to a subject that does not receive administration of the toxin serotype A while undergoing open-chest CABG surgery, and wherein the one or more atrial fibrillation episodes have a duration of equal to or greater than 30 seconds, equal to or greater than 30 minutes, equal to or greater than 4 hours, or combinations thereof. In some embodiments, the one or more post-operative atrial fibrillation episodes have a duration equal to or greater than 30 seconds, equal to or greater than 2 minutes, equal to or greater than 5 minutes, or combinations thereof. In some embodiments, the one or more post-operative atrial fibrillation episodes have a duration equal to or greater than 30 seconds, equal to or greater than 1 hour, and equal or greater than 6 hours, or combinations thereof. In some embodiments, the administering reduces the occurrence or likelihood of one or more post-operative atrial fibrillation episodes by at least 40%. In some embodiments, the administering reduces the occurrence or likelihood of one or more post-operative atrial fibrillation episodes by at least 50%.

In some embodiments, the methods further comprise selecting a subject that has a history or paroxysmal atrial fibrillation or a history of persistent atrial fibrillation. In some other embodiments, the methods further comprise selecting a subject that does not have a history or paroxysmal atrial fibrillation or a history of persistent atrial fibrillation.

Other benefits of the methods of the disclosure include reducing long-term clinical burden for subjects that have undergone open-chest CABG surgery, such as reducing length of stay in intensive care unit (ICU), reducing overall hospital length of stay, and reducing anticoagulant usage (e.g., increasing time period from surgery until usage of anticoagulant is necessary and/or reducing the amount of anticoagulant used).

In some embodiments, the administration of botulinum toxin serotype A to the epicardial fat pads during open-chest CABG surgery further results in a reduction in length of stay in intensive care unit (ICU) by at least 8 hours, 0.5 days, 1 day, 2 days, 3 days, 4 days, 5 days, or 6 days as compared to a subject that does not receive administration of the botulinum toxin serotype A while undergoing open-chest CABG surgery.

In some embodiments, the administering of the botulinum toxin serotype A further results in a reduction in length of stay of the subject in intensive care unit (ICU) by at least 8 hours, as compared to a subject that does not receive administration of the botulinum toxin serotype A while undergoing open-chest CABG surgery. In some embodiments, the administering of the botulinum toxin serotype A results in a reduction in length of stay of the subject in intensive care unit (ICU) by at least 0.5 days, as compared to a subject that does not receive administration of the botulinum toxin serotype A while undergoing open-chest CABG surgery. In some embodiments, the administering of the botulinum toxin serotype A results in a reduction in length of stay of the subject in intensive care unit (ICU) by at least 1 day, as compared to a subject that does not receive administration of the botulinum toxin serotype A while undergoing open-chest CABG surgery. In some embodiments, the administering of the botulinum toxin serotype A results in a reduction in length of stay of the subject in intensive care unit (ICU) by at least 2 days, as compared to a subject that does not receive administration of the botulinum toxin serotype A while undergoing open-chest CABG surgery. In some embodiments, the administering of the botulinum toxin serotype A results in a reduction in length of stay of the subject in intensive care unit (ICU) by at least 3 days, as compared to a subject that does not receive administration of the botulinum toxin serotype A while undergoing open-chest CABG surgery. In some embodiments, the administering of the botulinum toxin serotype A results in a reduction in length of stay of the subject in intensive care unit (ICU) by at least 4 days, as compared to a subject that does not receive administration of the botulinum toxin serotype A while undergoing open-chest CABG surgery. In some embodiments, the administering of the botulinum toxin serotype A results in a reduction in length of stay of the subject in intensive care unit (ICU) by at least 5 days, as compared to a subject that does not receive administration of the botulinum toxin serotype A while undergoing open-chest CABG surgery.

In some embodiments, the administration of botulinum toxin serotype A according to the methods described herein further reduces hospital length of stay by at least 0.5 days, 1 day, 2 days, 3 days, 4 days, 5 days, 6 days, 7 days, 8 days, 9 days, 10 days, 15 days, or 20 days as compared to a subject that does not receive administration of the botulinum toxin serotype A while undergoing open-chest CABG surgery. In some embodiments, the administration of botulinum toxin serotype A reduces hospital length of stay by at least 0.5 days. In alternative embodiments, the administration of botulinum toxin serotype A reduces hospital length of stay by at least 1 day.

In some embodiments, the administering of the botulinum toxin serotype A to the subject further reduces anticoagulant usage by at least 10% during a period of 30 days from administration, as compared to a subject that does not receive administration of the toxin serotype A while undergoing open-chest CABG surgery. In some embodiments, the administering of the botulinum toxin serotype A to the subject reduces anticoagulant usage by at least 20% during a period of 30 days from administration, as compared to a subject that does not receive administration of the toxin serotype A while undergoing open-chest CABG surgery. In some embodiments, the administering of the botulinum toxin serotype A to the subject reduces anticoagulant usage by at least 30% during a period of 30 days from administration, as compared to a subject that does not receive administration of the toxin serotype A while undergoing open-chest CABG surgery. In some embodiments, the administering of the botulinum toxin serotype A to the subject reduces anticoagulant usage by at least 40% during a period of 30 days from administration, as compared to a subject that does not receive administration of the toxin serotype A while undergoing open-chest CABG surgery. In some embodiments, the administering of the botulinum toxin serotype A to the subject reduces anticoagulant usage by at least 50% during a period of 30 days from administration, as compared to a subject that does not receive administration of the toxin serotype A while undergoing open-chest CABG surgery. In some embodiments, reducing anticoagulant usage comprises increasing the time to first anticoagulant use after surgery, reducing the amount of anticoagulant use, or both.

In some embodiment, the administering of the botulinum toxin serotype A to the subject reduces the occurrence or likelihood of one or more post-operative atrial fibrillation episodes by at least 30% during a period of 30 days from administration, as compared to a subject that does not receive administration of the toxin serotype A while undergoing open-chest CABG surgery, and wherein the one or more atrial fibrillation episodes have a duration of equal to or greater than 30 seconds, equal to or greater than 30 minutes, equal to or greater than 4 hours, or combinations thereof. In some embodiments, the one or more post-operative atrial fibrillation episodes have a duration equal to or greater than 30 seconds, equal to or greater than 2 minutes, equal to or greater than 5 minutes, or combinations thereof. In some embodiments, the one or more post-operative atrial fibrillation episodes have a duration equal to or greater than 30 seconds, equal to or greater than 1 hour, and equal or greater than 6 hours, or combinations thereof. In some embodiments, the administering reduces the occurrence or likelihood of one or more post-operative atrial fibrillation episodes by at least 40%. In some embodiments, the administering reduces the occurrence or likelihood of one or more post-operative atrial fibrillation episodes by at least 50%.

In some embodiments, the administering of the botulinum toxin serotype A to the subject reduces the occurrence or likelihood of one or more post-operative atrial fibrillation episodes by at least 40% during a period of 30 days from administration, as compared to a subject that does not receive administration of the toxin serotype A while undergoing open-chest CABG surgery, and wherein the one or more atrial fibrillation episodes have a duration of equal to or greater than 30 seconds, equal to or greater than 30 minutes, equal to or greater than 4 hours, or combinations thereof. In some embodiments, the administering further reduces hospital length of stay by at least 1 day. In some embodiments, the administering further reduces length of stay in the ICU by at least 0.5 days. In some embodiments, the administering further reduces the occurrence or likelihood of hospital readmission by at least 30%. In some embodiment, the administering further reduces anticoagulant usage by at least 30%.

In some embodiments, the subject has received a beta-blocker therapy before the open-chest CABG surgery and is withdrawn from the beta-blocker therapy after the open-chest CABG therapy. Beta blocker therapy is standard of care for patients getting cardiac surgery, especially CABG patients having lower heart rates and blood pressures. After cardiac surgery, for some patients, beta blocker therapy is withdrawn because of various medical reasons, including for example high blood loss, or low heart rates. The withdrawal of beta blocker therapy increases the incidences of post-operative atrial fibrillation in these patients, and therefore subjects withdrawn from the beta-blocker therapy are of an increased risk group.

In some embodiments, the administering of the botulinum toxin serotype A to the subject withdrawn from the beta-blocker therapy reduces the likelihood or occurrence of one or more post-operative atrial fibrillation episodes having a duration of equal to or greater than 30 seconds by at least 20% during a period of 30 days from administration, as compared to a subject that does not receive administration of the toxin serotype A while undergoing open-chest CABG surgery. In some embodiments, the administering of the botulinum toxin serotype A to the subject withdrawn from the beta-blocker therapy reduces the likelihood or occurrence of one or more post-operative atrial fibrillation episodes having a duration of equal to or greater than 30 seconds by at least 30% during a period of 30 days from administration, as compared to a subject that does not receive administration of the toxin serotype A while undergoing open-chest CABG surgery. In some embodiments, the administering of the botulinum toxin serotype A to the subject withdrawn from the beta-blocker therapy reduces the likelihood or occurrence of one or more post-operative atrial fibrillation episodes having a duration equal to or greater than 30 seconds by at least 40% during a period of 30 days from administration, as compared to a subject that does not receive administration of the toxin serotype A while undergoing open-chest CABG surgery. In some embodiments, the administering of the botulinum toxin serotype A to the subject withdrawn from the beta-blocker therapy reduces the likelihood or occurrence of one or more post-operative atrial fibrillation episodes having a duration equal to or greater than 30 seconds by at least 50% during a period of 30 days from administration, as compared to a subject that does not receive administration of the toxin serotype A while undergoing open-chest CABG surgery. In some embodiments, the one or more post-operative atrial fibrillation episodes have a duration equal to or greater than 30 seconds is selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours during a period of 30 days from administration.

In some embodiments, the one or more post-operative atrial fibrillation episodes have a duration equal to or greater than 5 minutes. In some embodiments, the one or more post-operative atrial fibrillation episodes have a duration equal to or greater than 30 minutes. In some embodiments, the one or more post-operative atrial fibrillation episodes have a duration equal to or greater than 1 hour. In some embodiments, the one or more post-operative atrial fibrillation episodes have a duration equal to or greater than 4 hours.

In certain aspects, the methods of the instant disclosure reduce anticoagulant usage after open-chest CABG surgery in a subject in need thereof.

In some embodiments, reducing anticoagulant usage comprises delaying the time to first anticoagulant use during a period of 30 days after open-chest CABG surgery. In some embodiments, reducing anticoagulant usage comprises reducing the amount of anticoagulant used during a period of 30 days after open-chest CABG surgery. In some embodiments, reducing anticoagulant usage comprises both delaying the time to first anticoagulant use and reducing the amount of anticoagulant used during a period of 30 days after open-chest CABG surgery.

In some embodiments, the administration of botulinum toxin serotype A according to the methods described herein reduces anticoagulant usage during the first 30 days from administration (i.e., after the administering) by at least 10% as compared to a subject that does not receive administration of the botulinum toxin serotype A while undergoing open-chest CABG surgery.

In some embodiments, the administering of the botulinum toxin serotype A to the subject reduces anticoagulant usage by at least 10% during a period of 30 days from administration, as compared to a subject that does not receive administration of the toxin serotype A while undergoing open-chest CABG surgery. In some embodiments, the administering of the botulinum toxin serotype A to the subject reduces anticoagulant usage by at least 20% during a period of 30 days from administration, as compared to a subject that does not receive administration of the toxin serotype A while undergoing open-chest CABG surgery. In some embodiments, the administering of the botulinum toxin serotype A to the subject reduces anticoagulant usage by at least 30% during a period of 30 days from administration, as compared to a subject that does not receive administration of the toxin serotype A while undergoing open-chest CABG surgery. In some embodiments, the administering of the botulinum toxin serotype A to the subject reduces anticoagulant usage by at least 40% during a period of 30 days from administration, as compared to a subject that does not receive administration of the toxin serotype A while undergoing open-chest CABG surgery. In some embodiments, the administering of the botulinum toxin serotype A to the subject reduces anticoagulant usage by at least 50% during a period of 30 days from administration, as compared to a subject that does not receive administration of the toxin serotype A while undergoing open-chest CABG surgery. In some embodiments, reducing anticoagulant usage comprises increasing the time to first anticoagulant use after surgery, reducing the amount of anticoagulant use, or both.

In some embodiments, the administering of the botulinum toxin serotype A to the subject increases the time to first anticoagulant use after surgery by at least 10% during a period of 30 days from administration, as compared to a subject that does not receive administration of the toxin serotype A while undergoing open-chest CABG surgery. In some embodiments, the administering of the botulinum toxin serotype A to the subject increases the time to first anticoagulant use after surgery by at least 20% during a period of 30 days from administration, as compared to a subject that does not receive administration of the toxin serotype A while undergoing open-chest CABG surgery. In some embodiments, the administering of the botulinum toxin serotype A to the subject increases the time to first anticoagulant use after surgery by at least 30% during a period of 30 days from administration, as compared to a subject that does not receive administration of the toxin serotype A while undergoing open-chest CABG surgery. In some embodiments, the administering of the botulinum toxin serotype A to the subject increases the time to first anticoagulant use after surgery by at least 40% during a period of 30 days from administration, as compared to a subject that does not receive administration of the toxin serotype A while undergoing open-chest CABG surgery. In some embodiments, the administering of the botulinum toxin serotype A to the subject increases the time to first anticoagulant use after surgery by at least 50% during a period of 30 days from administration, as compared to a subject that does not receive administration of the toxin serotype A while undergoing open-chest CABG surgery.

In some embodiments, the administering of the botulinum toxin serotype A to the subject delays the need for an anticoagulant by at least 1 day, at least 2 days, at least 3 days, at least 4 days, at least 5 days, at least 6 days, at least 7 days, at least 8 days, at least 9 days or at least 10 days as compared to a subject that does not receive administration of the toxin serotype A while undergoing open-chest CABG surgery.

Co-administration of botulinum toxin serotype A with a second agent should only performed with caution due to potential drug-drug interactions leading to unwanted effects. Co-administration with a second agent interfering with neuromuscular transmission (e.g., a curare-like compounds, an aminoglycoside) should only be performed with caution as the effect of the toxin may be potentiated. Use of an anticholinergic drug after administration of the botulinum toxin may potentiate systemic anticholinergic effects. Excessive muscle weakness may also be exaggerated by administration of a muscle relaxant before or after administration of the botulinum toxin.

Therefore, in some embodiments of the methods described herein, the botulinum toxin serotype A is not co-administered (e.g., before, at the same time, or after) with a second agent selected from the group consisting of a drug that interferes with neuromuscular transmission, an aminoglycoside, an anticholinergic drug, or a muscle relaxant.

### Administration of Botulinum Toxin During Open-Heart Surgery

Methods of preventing post-operative atrial fibrillation and/or preventing hospital readmission (i.e., rehospitalization) after discharge after open-chest CABG surgery in a subject in need thereof are provided.

In some embodiments, the methods provided herein comprise administering (e.g., injecting) a neuromodulating substance (e.g., a botulinum toxin) to the epicardial fat pads. The neuromodulating substance can be clostridial toxins (e.g., botulinum toxins), antiarrhythmic medications, and/or nerve blocking agents. In some embodiments, the neuromodulating substance is botulinum toxin serotype A.

The methods comprise administering to one or more epicardial fat pads of the subject who is undergoing an open-heart cardiac surgery an effective amount of a botulinum toxin. The epicardial fat pads include aortic fat pad, two right-side pulmonary vein fat pads, and two left-side pulmonary vein fat pads.

During open-chest CABG surgery, sometimes also referred to as open-heart cardiac surgery, these epicardial fat pads can be readily visualized by the operating surgeon with minimal further manipulation of the heart's position. In addition, these epicardial fat pads could be administered with a neuromodulating substance with little extension of operative time, cardiopulmonary bypass time, or cardioplegia time; this is in contrast to performing intra-operative ablations of cardiac tissue which can significantly increase operative, bypass, and cardioplegia times.

In contrast to some known methods of administration of botulinum toxin to reduce atrial fibrillation after cardiac surgery, in some embodiments of the methods of this disclosure, the botulinum toxin composition is administered before the primary procedure (such as such as coronary artery bypass grafting or valve repair/replacement) or concurrently with the primary procedure.

For example, a clinical study described in “Long-Term Suppression of Atrial Fibrillation by Botulinum Toxin Injection into Epicardial Fat Pads in Patients Undergoing Cardiac Surgery: One Year Follow Up of a Randomized Pilot Study” by Pokushalov et al., Circulation Arrhythmia and Electrophysiology, Oct. 25, 2015 (DOI: 10.1161/CIRCEP.115.003199) administered botulinum toxin during coronary artery bypass graft (CABG) surgery into four epicardial fat pads instead of five and specifically after the main stage of surgery. The four fat pads included (1) left atrial fat pad located anterior to the right superior pulmonary vein and corresponded to the anterior right ganglionated plexi (GP), (2) the fat pad located infero-posterior to the right inferior pulmonary vein and corresponded to the inferior right GP, (3) the fat pad located anterior to the left superior PV and left inferior pulmonary vein (PV) and corresponded to the Marshall tract GP and superior left GP, and (4) the fat pad located inferiorly to the left inferior PV and extended posteriorly and corresponded to the inferior left GP.

Another clinical study described in “Temporary autonomic modulation with botulinum toxin type A to reduce atrial fibrillation after cardiac surgery” by Waldron et al., Heart Rhythm, pp. 1-7, 2018, administered onabotulinumtoxinA into five epicardial fat pads immediately after initiation of cardiopulmonary bypass, before the primary surgical procedure. The five fat pads include the anterior fat pad, and the fat pads associated with each pulmonary vein.

In some embodiments of the present method, when the patient is undergoing off-bypass surgery, the botulinum toxin composition is administered while the patient is on off-pump bypass before the primary procedure.

In some embodiments, when the patient is undergoing on-bypass surgery, the botulinum toxin composition is administered prior to initiation of cardiopulmonary bypass, while the subject is on cardiopulmonary bypass, and/or after cardiopulmonary bypass. For subjects undergoing cardiopulmonary bypass, it is important to limit time on cardiopulmonary bypass. In some embodiments, the botulinum toxin composition is administered: (i) before cardiopulmonary bypass is initiated (for example before placement of bypass cannulas), (ii) while bypass cannulas are being placed on the subject, and/or (iii) while the subject is on cardiopulmonary bypass. In some embodiments, the botulinum toxin composition is administered before bypass cannulas are placed on the subject. In some embodiments, the botulinum toxin composition is administered while bypass cannulas are being placed on the subject. In some embodiments, the botulinum toxin composition is administered while the subject is on cardiopulmonary bypass. In some embodiments, in the cases where cardiac arrest is to be induced, the botulinum toxin composition is administered: (i) before cross-clamping, (ii) after cross-clamping has been performed, and/or (iii) after cardioplegia has been instituted.

For both off-pump and on-pump surgeries, injections of botulinum toxin composition are done prior to the primary surgical procedure to avoid disturbing any of the planned surgery work (e.g., coronary artery bypass grafting, valve placement). If the heart is to be arrested, the botulinum toxin composition can also be administered after induction of cardiac arrest. In an embodiment, the botulinum toxin composition is not administered into the coronary arteries or coronary sinus, particularly during administration to the left side pulmonary vein fat pad pads.

During open-heart cardiac surgery, a sternotomy is performed to fully access the heart. The pericardial sac is then dissected for the heart to be further accessible for the procedure. At this point of cardiac surgery, the subject may or may not be put onto cardiopulmonary bypass, depending on the type of surgery (e.g., on- or off-bypass) and particulars of the case and the subject. Administration of a botulinum toxin composition to an administration site on the epicardial fat pads may be performed at this early time in the surgical procedure; the rationale of this timing is to have early drug administration and to avoid later interference with newly operated areas.

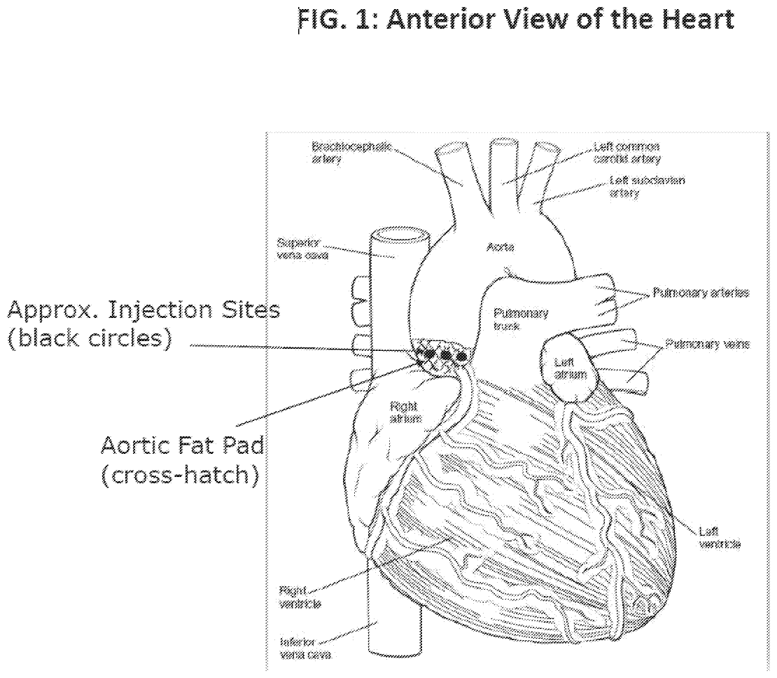

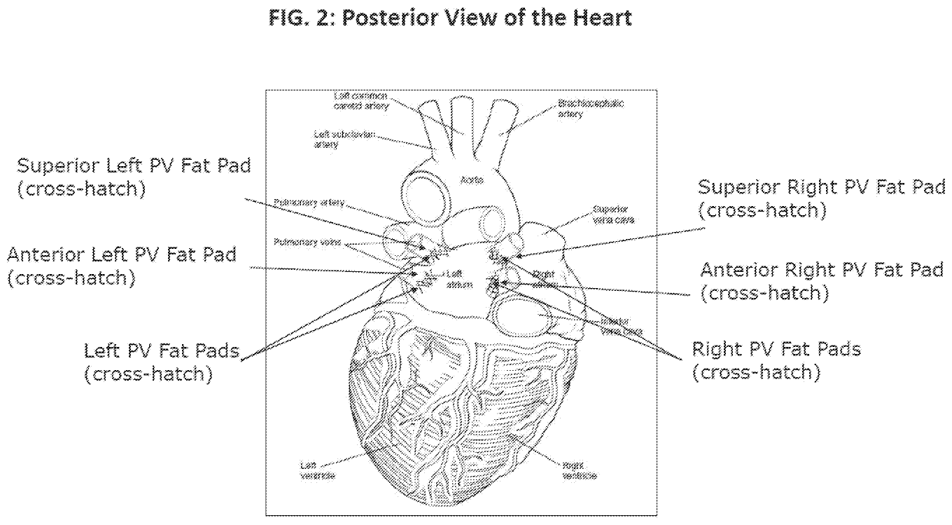

The aortic fat pad stretches across the epicardial region at the base of the aorta, forming a discrete, thin band of tissue approximately 1-2 mm thick (FIG. 1). The two right-side pulmonary vein epicardial fat pads can be observed when the heart is shifted to the subject's left side (FIG. 2). These epicardial fat pads are located at the base of each respective pulmonary vein (inferior and superior) near or at the junction of the pulmonary vein and atria. The two left-side pulmonary vein epicardial fat pads can be observed when the heart is shifted to the subject's right side (FIG. 2). These epicardial fat pads are located near or at the base of each respective pulmonary vein (inferior and superior) near the junction of the pulmonary vein and atria. In addition to these five distinct fat pad regions, the ligament of Marshall contains dense nerve clusters and may also be identified.

In some embodiments, the method describes herein anatomically locates an administration site on the epicardial fat pads. In one embodiment, the administration site comprises the ganglionic plexi residing on the epicardial fat pads. In an embodiment, the method described herein does not comprise electrically stimulating the epicardial fat pads to locate an administration site on the epicardial fat pads.

Locating the ganglionic plexi, anatomically is superior to locating them by electrical stimulation at least because the anatomical locating or targeting (1) is much less time-consuming during a portion of the surgical procedure in which time efficiency is critical, and (2) does not require extensive electrophysiological equipment for probing sites, providing high-frequency electrical stimulation, and recording and storing the resultant outputs of cardiac parameters. Moreover, electric stimulation may require additional manipulation of anatomic structures within the chest cavity and on the surgical field. Furthermore, the goal of electrical targeting is usually to obtain an evoked response in cardiac and blood pressure parameters.

Accordingly, in some embodiments, the method disclosed herein does not comprise electrically stimulating the epicardial fat pads to locate an administration site on the epicardial fat pads. That is, the administering to the epicardial fat pads is performed in the absence of electrical stimulation and the administration sites are determined anatomically. In some embodiments, the administering to the epicardial fat pads is not preceded by electrical stimulation nor done concurrently with electrical stimulation.

### A. Aortic Fat Pad

Aortic fat pad is also known as the ventral fat pad or anterior fat pad. This fat pad stretches across the base of the aorta and is approximately 1-2 mm thick. Preferably, botulinum toxin is administered to this aortic fat pad first because of its proximity to the opening in the chest and one of the first epicardial surfaces encountered upon opening of the pericardial sac. Little to no manipulation of the heart is needed to gain access to this fat pad.

In some embodiments, the administration can be readily performed prior to initiation of cardiopulmonary bypass (i.e., prior to the insertion of the cannulas). In some embodiments, the botulinum toxin composition is administered to the aortic fat pad while bypass cannulas are being placed on the subject. In some embodiments, the botulinum toxin composition is administered while the subject is on cardiopulmonary bypass. In some embodiments, the botulinum toxin composition is administered to the aortic fat pad before the primary surgery and: (i) before cardiopulmonary bypass is initiated, (ii) while bypass cannulas are being placed on the subject, and/or (iii) while the subject is on cardiopulmonary bypass. In some embodiments, the botulinum toxin composition is administered to the aortic fat pad after cardiopulmonary bypass and before the primary surgery. In some embodiments, if cardiac arrest is to be induced, the botulinum toxin composition can also be administered to the aortic fat pad: (i) before cross-clamping, (ii) after cross-clamping has been performed, and/or (iii) after cardioplegia has been instituted. If the subject is undergoing off-pump bypass, the administration can be performed while the subject is on off-pump bypass before the primary procedure.

Depending on the amount of botulinum toxin to be administered, it may be necessary to make more than one administration site in different locations in the region of this fat pad. For example, if 1 mL of botulinum toxin is to be administered into this fat pad, the 1 mL volume can be deposited across 1, 2, 3, 4, or 5 administration sites. In one embodiment, the amount of botulinum toxin is administered across 1-3 administration sites corresponding, wherein the amount is apportioned across the 1-3 sites.

In some embodiments, the administration of botulinum toxin is via injection with a syringe which has a needle. The needle gauge can be 25-30 in order to limit the puncture size, both to prevent leakage of botulinum toxin and to avoid unnecessary bleeding and tissue disruption. The needle length can range from 0.5 inch to 3.5 inch. Longer needles (e.g., spinal needles with needle length up to 3.5 inch) can also be used for these injections, depending on the depth of the epicardial target within the opened chest cavity. Minimization of the number of punctures can reduce chance of leakage and tissue trauma.

In some embodiments, it may be useful to perform a needle bend (approximately 10-90 degrees such as 60-80 degrees) in order to most readily achieve an efficient angle of approach to this target site to deposit the botulinum toxin. Bending the needle can make the injection much quicker as the needle can clearly be seen under the epicardial fat without concern of puncturing the aorta.

Upon insertion of the needle into this fat pad, the syringe may or may not be drawn back to ensure that the tip of the needle is not inside the aorta. This approach is not necessarily needed if the injection is performed after the heart and lower aorta have been drained of blood; however, it can still be performed to test for air pockets.

As the botulinum toxin is injected, a wheal (swelling of epicardial fat tissue, as it visibly enlarges with injected substance) is clearly observed which indicates that the proper target was successfully injected. Minimal to no leakage of injected substance should be observed.

Use of forceps to grip and secure the syringe-needle juncture may be useful for guiding the needle tip to the proper target. After the botulinum toxin is completely injected, the syringe and needle can be disposed.

### B. Two Right-Side Pulmonary Vein Fat Pads

These two fat pads are at or near the junction of each right-side pulmonary vein (superior and inferior) and the atria. Preferably, the botulinum toxin is administered to these two fat pads after the aortic fat pad administration because of the ease of access to the right-sided pulmonary vein fat pads, particularly if the operating surgeon is at the subject's right side.

The administration can be performed prior to initiation of cardiopulmonary bypass (i.e., prior to the insertion of the cannulas), during cardiopulmonary bypass, or after cardiopulmonary bypass. In some embodiments, the botulinum toxin composition is administered to the right-side pulmonary vein fat pads while bypass cannulas are being placed on the subject. In some embodiments, the botulinum toxin composition is administered while the subject is on cardiopulmonary bypass. In some embodiments, the botulinum toxin composition is administered to the right-side pulmonary vein fat pads before the primary surgery and: (i) before cardiopulmonary bypass is initiated, (ii) while bypass cannulas are being placed on the subject, and/or (iii) while the subject is on cardiopulmonary bypass. In some embodiments, the botulinum toxin composition is administered to the right-side pulmonary vein fat pads after cardiopulmonary bypass and before the primary surgery. In some embodiments, if cardiac arrest is to be induced, the botulinum toxin composition can also be administered to the right-side pulmonary vein fat pads: (i) before cross-clamping, (ii) after cross-clamping has been performed, and/or (iii) after cardioplegia has been instituted. The latter circumstance will allow the injection-target to be more static and efficiently approached. If the subject is undergoing off-pump coronary artery bypass rather than cardiopulmonary bypass, the administration can be performed while the subject is undergoing off-pump coronary artery bypass.

Depending on the amount of the botulinum toxin to be administered, it may be necessary to make more than one administration sites in different locations in the region of each of the two fat pads. For example, if 1 mL of botulinum toxin is to be administered to one of the two fat pads, the 1 mL volume can be deposited across 1, 2, 3, 4, or 5 administrations (e.g., spreading out the 1 mL across the fat pad and in slightly different angulations). However, the particular fat pads described herein are very accommodating to at least 1 mL of deposited substance. The number of administration sites per fat pad should be minimized if possible, as to attenuate the chance of leakage.

Each of the two right-side pulmonary vein fat pads can be injected with a single injection of appropriate volume, e.g., 1 mL, of botulinum toxin and not distributed across multiple injections. Spreading injections across pulmonary vein fat pads may lead to leakage. Injections should be made at a depth of 1-2 mm. Minimization of the number of punctures can also reduce chance of leakage and tissue trauma.

In some embodiments, the administration of botulinum toxin is via injection with a syringe which has a needle. The needle gauge should be 25-30 in order to limit the puncture size, both to prevent leakage of the botulinum toxin and to avoid unnecessary tissue disruption.

The needle length can range from 0.5 inch to 3.5 inch. Longer needles (e.g., spinal needles with length up to 3.5 inch) can also be used for these injections.

For this injection, it may be useful to perform a slight needle bend (approximately. 10-90 degrees) in order to most readily achieve an efficient angle of approach to this fat pad to properly deposit the botulinum toxin. Bending the needle can make the injection much quicker as the needle can clearly be seen under the epicardial fat.

Using forceps to guide the needle can be useful to perform these injections. Upon insertion of the needle into the epicardial fat pad, the syringe may or may not be drawn back to ensure that the tip of the needle is not inside the pulmonary vein or atria. This approach is not necessarily needed if the injection is performed after the heart has been drained of blood; however, it can still be performed to test for air pockets.

As the botulinum toxin is injected, a wheal (swelling of epicardial fat tissue, as it visibly enlarges with injected substance) is often clearly observed which indicates that the proper fat pad was successfully injected. Minimal to no leakage of injected substance should be observed.

After the botulinum toxin is completely injected, the syringe and needle can be disposed.

### C. Two Left-Side Pulmonary Vein Fat Pads

These fat pads are at or near the junction of each left-side pulmonary vein (superior and inferior) and the atria. Preferably, the botulinum toxin is administered to these two fat pads last because of their deeper and more posterior location in the chest cavity. The heart must be shifted to the right to expose the left superior fat pad and the left anterior fat pad. Additionally, the left atrial appendage often must be retracted to access the left superior fat pad, and the apex of the heart may require retraction to access the left inferior fat pad.

The administration of the botulinum toxin to the left-side pulmonary vein fat pads can be performed prior to initiation of cardiopulmonary bypass (i.e., prior to the insertion of the cannulas), during cardiopulmonary bypass, or after cardiopulmonary bypass. In some embodiments, the botulinum toxin composition is administered to the left-side pulmonary vein fat pads while bypass cannulas are being placed on the subject. In some embodiments, the botulinum toxin composition is administered while the subject is on cardiopulmonary bypass. In some embodiments, the botulinum toxin composition is administered to the left-side pulmonary vein fat pads before the primary surgery and: (i) before cardiopulmonary bypass is initiated, (ii) while bypass cannulas are being placed on the subject, and/or (iii) while the subject is on cardiopulmonary bypass. In some embodiments, the botulinum toxin composition is administered to the left-side pulmonary vein fat pads after cardiopulmonary bypass and before the primary surgery. In some embodiments, if cardiac arrest is to be induced, the botulinum toxin composition can also be administered to the rig-side pulmonary vein fat pads: (i) before cross-clamping, (ii) after cross-clamping has been performed, and/or (iii) after cardioplegia has been instituted. The latter circumstance will allow the injection-target to be more static and efficiently approached.

These fat pads are typically administered while on-pump, but prior to cross-clamp; however, some surgeons may opt to perform the left-side injections after cardioplegia, especially if the heart is beating rapidly or the left atrial appendage (LAA) is obstructing the injection. If the subject is undergoing off-pump bypass, the administration can be performed while the subject is undergoing off-pump bypass.

Depending on the amount of the botulinum toxin to be administered, it may be necessary to make more than one administration in different locations in the region of each of the two fat pads. For example, if 1 mL of the botulinum toxin is to be administered into one of the two fat pads, the 1 mL volume can be deposited across 1, 2, 3, 4, or 5 administrations (e.g., spreading out the 1 mL across the length of each of the two fat pads and in slightly different angulations). However, the particular fat pads described herein are very accommodating to at least 1 mL of deposited substance. The number of administration sites per fat pad should be minimized if possible, as to attenuate the chance of leakage).

Each of the two left-side pulmonary vein fat pads can be injected with a single injection of appropriate volume, e.g., 1 mL, of botulinum toxin and not distributed across multiple injections. Spreading injections across pulmonary vein fat pads may lead to leakage. Injections should be made at a depth of 1-2 mm. Minimization of the number of punctures can reduce chance of leakage and tissue trauma.

In some embodiments, administration of botulinum toxin is via injection with a syringe which has a needle. The needle gauge should be 25-30 in order to limit the puncture size, both to prevent leakage of the botulinum toxin and to avoid unnecessary tissue disruption.

The needle length can range from 0.5 inch to 3.5 inch. In some cases, a spinal needle with length up to 3.5 inch may also be considered, especially in subjects with a deep chest or when the size of the opening in the chest is limited. The utility of a longer needle depends on the depth of the epicardial fat pads within the opened chest cavity and has been observed to be even more useful for the left-sided fat pad targets, which are deep and posterior relative to other targets.

For this injection, it may be useful to perform a slight needle bend (approximately 10-90 degrees) to most readily achieve an efficient angle of approach to this target site to properly deposit the botulinum toxin. Bending the needle can make the injection much quicker as the needle can clearly be seen under the epicardial fat.

Using forceps to guide the needle can be useful to perform these injections. Upon insertion of the needle into the epicardial fat pads, the syringe may or may not be drawn back to ensure that the tip of the needle is not inside the pulmonary vein or atria. This approach is not necessarily needed if the administration is performed after the heart has been drained of blood; however, it can still be performed to test for air pockets.

As the botulinum toxin is injected, a wheal (swelling of epicardial fat tissue, as it visibly enlarges with injected substance) is often clearly observed which indicates that the proper target was successfully injected. Minimal to no leakage of injected substance should be observed.

After the botulinum toxin is completely injected, the syringe and needle can be disposed.

The order of injections of the left-sided inferior and superior fat pads, in some embodiments, is irrelevant.

### D. Administration Techniques

In the methods disclosed herein, the step of administering can be via injection or topically. In one embodiment, administration is via injection into each epicardial fat pad. For example, a composition comprising the effective amount of botulinum toxin is administered at a single injection site in each epicardial fat pad by insertion of an appropriately sized needle (e.g., 0.5-3.5-inch needle) at a needle penetration point. Once the needle of the syringe is inserted at a needle penetration point, it is then inserted to its full or any appropriate length (or less than its full length, if so desired) and then once at the end of its insertion a portion of the composition comprising the botulinum toxin is delivered to the injection site by depressing the plunger of the syringe to deliver the desired dose at that injection site. The needle is then withdrawn and once again inserted to its full or any appropriate length (or less than its full length, if so desired) at the next injection site, where an amount of the composition comprising an amount of the toxin is delivered at the injection site.

It is contemplated that the time needed to perform the total injections—ranges, in some embodiments, from 1-5 minutes, 1-6 minutes, 1-7 minutes, 1-8 minutes, 1-9 minutes, 1-10 minutes, 2-10 minutes, 2-9 minutes, 2-8 minutes, 2-7 minutes, 3-10 minutes, 3-9 minutes, 3-8 minutes, 3-7 minutes, 3-6 minutes, 4-10 minutes, 4-9 minutes, 4-8 minutes, 4-7 minutes, 4-6 minutes, 5-10 minutes, 5-9 minutes, 5-8 minutes, 5-7 minutes, or 5-6 minutes. In some embodiment, the time needed to perform the total injections is about 5 minutes. In some embodiments, the injection time anticipated is 10-15 seconds for each injection, or 10-45 seconds total per fat pad. Injections should be made at a depth of 1-2 mm, depending on the thickness of the fat pad. In some cases, injections at a depth of >2 mm can be conducted. Angles of injection can vary, depending on subject anatomy and include injections which a perpendicular to the fat pad (i.e., direct injections), injections which are slightly angled, and injections at an oblique angle. It will be appreciated that a short time to complete the total injections reduces bypass time, reduces chances for adverse effects, and increases safety for subjects.

The method described herein provides certain advantages over previously known methods wherein botulinum toxin is injected to epicardial fat pads, including shorter injection time, allowing the toxin to become effective as soon as possible after surgery, reduced bypass time, reduced chance for leakage and tissue trauma, reduced risk of medical complications and increased safety for subjects. For example, the method described herein minimizes the total treatment (administration) time, where in an embodiment, the total time for administration of the effective dose is between about from 1-5 minutes, 1-6 minutes, 1-7 minutes, 1-8 minutes, 1-9 minutes, 1-10 minutes, 2-10 minutes, 2-9 minutes, 2-8 minutes, 2-7 minutes, 3-10 minutes, 3-9 minutes, 3-8 minutes, 3-7 minutes, 3-6 minutes, 4-10 minutes, 4-9 minutes, 4-8 minutes, 4-7 minutes, 4-6 minutes, 5-10 minutes, 5-9 minutes, 5-8 minutes, 5-7 minutes, or 5-6 minutes. For comparison, previously known methods have a total injection time of about 10-11 or of greater than about 7 minutes or of greater than 5-10 minutes. In some embodiments, the administration of the botulinum toxin to the epicardial fat pads is carried out in a specific order, wherein the aorta fat pad is injected first, followed by injection into the pulmonary vein fat pads. In some embodiments, following toxin injection into the aorta fat pad, the botulinum toxin is administered to the right-side pulmonary vein fat pads. In some embodiments, the toxin is administered to the left-side pulmonary vein fat pads subsequent to the aorta fat pad injection. In one embodiment of the method described herein, the effective amount of toxin is administered by injection prior to initiation of a cardiac pulmonary bypass procedure, while the cannulas are being placed on the subject, and/or during the bypass procedure, whereas previously known methods administer toxin via injection after the primary surgery or after the bypass procedure. Having the injection done prior to initiation of cardiopulmonary bypass and/or during the bypass allows the toxin to become effective as soon as possible after surgery, have longer time to act, and minimizes the risk of damaging the needle work after the procedure is done. The method described herein has a minimal number of punctures per fat pad to reduce chance for leakage and tissue trauma whereas the previously known methods have multiple punctures into each fat pad (up to 5 or 6 per fat pad). That is, in one embodiment, a single puncture into each of the five epicardial fat pads is made in the performance of the method. Furthermore, the method described herein provides clear step by step guidance. In contrast, the previously known methods provide no specific guidance to the surgeons, resulting in a highly variable time range as different surgeons use difference approaches.

In some embodiments, the botulinum toxin is injected at 1-5 locations per fat pad. In some embodiments, the botulinum toxin is injected at 1, 2 or 3 locations per fat pad.

In some embodiments, the time for administering the botulinum toxin is about 5-30 seconds, or 10-15 seconds per injection.

In some embodiments, the time for administering the botulinum toxin is about 5-150 seconds, or 10-45 seconds per fat pad.

Studies were performed in support of the method disclosed herein. In a first patient case, detailed in Example 7, a patient undergoing an open-chest cardiac surgery had an effective amount of a botulinum toxin type A injected into the epicardial fat pads, where a first portion of the effective amount was injected into the aortic fat pad while the cannulas were being inserted for cardiopulmonary bypass. The remaining amount of the effective amount of the botulinum toxin type A was then injected into the four pulmonary vein fat pads while the patient was on bypass. The time for injection into the four pulmonary vein fat pads was approximately 3-4 minutes. The total time for injection into the five epicardial fat pads was approximately 5 minutes.

In Example 8, a patient undergoing an open-chest cardiac surgery was treated with a dose of a botulinum toxin type A via injection. To administer the dose of botulinum toxin into the aortic fat pad, the needle was ‘bent’ or angled relative to the treatment surface. The botulinum toxin was administered to the aortic fat pad before initiation of the cardiopulmonary bypass (i. e. prior to insertion of the cannulas). The remaining doses of botulinum toxin were administered to the four pulmonary vein fat pads while the patient was on cardiopulmonary bypass. The total time for administration of the botulinum toxin doses to the pulmonary vein fat pads was approximately 3-4 minutes. The total injection time to the five epicardial fat pads was about 5 minutes.

In another patient case, described in Example 9, a patient undergoing an open-chest cardiac surgery had an amount of a botulinum toxin type A administered via injection first to the aortic fat pad and then to the right-side pulmonary vein fats pads while the patient was on cardiopulmonary bypass. Forceps were used to guide the injection. Injections were also made into the left-side pulmonary vein fat pads using forceps.

As mentioned above, and as set forth in the patient case of Example 10, a clear wheal or swelling of the epicardial fat tissue is an indication of successful injection of the toxin.

In other cases, accommodations were made for physical attributes of the patient. For example, a study in Example 11 utilized a longer needle, such as a spinal needle, to administer the effective amount of botulinum toxin to the patient. The use of a longer needle minimized the need for manipulation of the heart in order to reach the appropriate injection target.

In Examples 1-6, a clinical study is described; this study was a multi-center, phase 2 randomized, double-blind, placebo-controlled, parallel group dose-ranging study to evaluate the efficacy and safety of botulinum toxin type A injection into the epicardial fat pads to prevent POAF in patients undergoing cardiac surgery. Patients were divided into three interventional arms randomized in a 1:1:1 fashion: 125 units of botulinum toxin type A (25 units per fat pad), 250 units of a botulinum toxin type A (50 units per fat pad), and placebo.

The injection paradigm used in the clinical trial described in Example 1 is distinguishable from the known methods. It includes, for example, performing the injections of botulinum toxin type A into the aortic fat pad first followed by the two right side pulmonary vein fat pads then the two left side pulmonary vein fat pads or followed by the two left side pulmonary vein fat pads then the two right side pulmonary vein fat pads; performing the injections of botulinum toxin type A into the five fat pads prior to the primary surgery (in on-pump surgeries the injections can be performed prior to cardiopulmonary bypass or while bypass cannulas are being placed on the patient to minimize pump time, and/or while the patient is on cardiopulmonary bypass), minimizing the number of punctures per fat pad to not more than three, and bending the needle used in the injections to most readily achieve an efficient angle.

The injection paradigm used in the clinical trial described in Example 1 has several advantages relative to the known methods. For example, by performing the injections of botulinum toxin type A before the primary surgery, including prior to initiation of the cardiopulmonary bypass, while bypass cannulas are being placed on the patient, during cardiopulmonary bypass, after cross-clamping, and/or after cardioplegia has been instituted, the present method allows the toxin to become effective soon after surgery and have a longer time to act, minimizes the risk of damaging the needle work after the procedure is done, avoids disturbing any of the coronary artery bypass grafting (CABG) and/or valve work. By minimizing the number of punctures per fat pad, the present method reduces the risk for leakage and tissue trauma. Overall, relative to the known methods, in addition to the above-mentioned advantages, the present injection paradigm reduces injection time, reduces bypass time, reduces length of surgery, increases patient safety and reduces risks for complications.

The study shows that injection of botulinum toxin type A into the epicardial fat pads reduced the occurrence of POAF, reduced the incidence of adverse outcomes known to be associated with POAF, favorably impacted healthcare resource utilization, and was well-tolerated in patients undergoing cardiac surgery.

The primary efficacy endpoint was the percentage of patients with at least one episode of atrial fibrillation (AF) of equal to or greater than about 30 seconds in the first 30 days after surgery. The secondary efficacy endpoints included measures of AF burden in the first 30 days after surgery, including the total percentage of time spent in AF, percentage of patients with at least one symptomatic AF event, time to occurrence of the first AF event after surgery, and the percentage of patients with at least one continuous episode of AF of greater than or equal to 2 minutes, 5 minutes, 6 minutes, 30 minutes, 1 hour, 4 hours, 6 hours, 12 hours, or 24 hours.

### Botulinum Neurotoxin

The genus Clostridium has more than one hundred and thirty species, grouped by morphology and function. The anaerobic, gram positive bacterium Clostridium botulinum produces a potent polypeptide neurotoxin, botulinum toxin (synonymously “toxin”), which causes a neuroparalytic illness in humans and animals known as botulism. Symptoms of botulinum toxin intoxication can progress from difficulty walking, swallowing, and speaking to paralysis of the respiratory muscles and death.

Non-limiting examples of Clostridial toxins include a Botulinum toxin like BoNT/A, a BoNT/B, a BoNT/C₁, a BoNT/D, a BoNT/E, a BoNT/F, a BoNT/G, a BoNT/H, a BoNT/X, a mosaic Botulinum toxin and/or subtypes and variants thereof.

Although all the botulinum toxins serotypes apparently inhibit release of the neurotransmitter acetylcholine at the neuromuscular junction, they do so by affecting different neurosecretory proteins and/or cleaving these proteins at different sites. Botulinum toxin type A is a zinc endopeptidase which can specifically hydrolyze a peptide linkage of the intracellular, vesicle-associated protein (VAMP, also called synaptobrevin) 25 kiloDalton (kDa) synaptosomal associated protein (SNAP-25). Botulinum type E also cleaves SNAP-25 but targets different amino acid sequences within this protein, as compared to botulinum toxin type A. Botulinum toxin types B, D, F and G act on VAMP with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes.

The molecular weight of the active botulinum toxin protein molecule (also known as “pure toxin” or as the “neurotoxic component”) from a botulinum toxin complex, for all of the known botulinum toxin serotypes, is about 150 kDa. Interestingly, the botulinum toxins are released by Clostridial bacterium as complexes comprising the 150 kDa neurotoxic component along with one or more associated non-toxin proteins. Thus, the botulinum toxin type A complex can be produced by Clostridial bacterium as 900 kDa, 500 kDa and 300 kDa forms (approximate molecular weights). Botulinum toxin types B and C₁ are apparently produced as only a 500 kDa complex. Botulinum toxin type D is produced as both 300 kDa and 500 kDa complexes. Finally, botulinum toxin types E and F are produced as only approximately 300 kDa complexes. The complexes (i.e., molecular weight greater than about 150 kDa) contain hemagglutinin (HA) proteins and a non-toxin non-hemagglutinin (NTNH) protein. Thus, a botulinum toxin complex can comprise a botulinum toxin molecule (the neurotoxic component) and one or more HA proteins and/or NTNH protein. These two types of non-toxin proteins (which along with the botulinum toxin molecule can comprise the relevant neurotoxin complex) may act to provide stability against denaturation to the botulinum toxin molecule and protection against digestive acids when toxin is ingested. Additionally, it is possible that the larger (greater than about 150 kDa molecular weight) botulinum toxin complexes may result in a slower rate of diffusion of the botulinum toxin away from a site of intramuscular injection of a botulinum toxin complex. The success of botulinum toxin type A to treat a variety of clinical conditions has led to interest in other botulinum toxin serotypes. Thus, at least botulinum toxin types, A, B, E and F have been used clinically in humans.

The botulinum toxin type A is known to be soluble in dilute aqueous solutions at pH 4-6.8. At pH above about 7 the stabilizing non-toxin proteins dissociate from the neurotoxin, resulting in a gradual loss of toxicity, particularly as the pH and temperature rise (Schantz E. J., et al Preparation and characterization of botulinum toxin type A for human treatment, chapter 3 of Jankovic, J., et al, Therapy with Botulinum Toxin, Marcel Dekker, Inc, 1994).

As with enzymes generally, the biological activities of the botulinum toxins (which are intracellular peptidases) are dependent, at least in part, upon their three-dimensional conformation. Dilution of the toxin from milligram quantities to a solution containing nanograms per milliliter presents significant difficulties, such as, for example, tendency for toxin to adhere to surfaces and thus reduce the amount of available toxin. Since the toxin may be used months or years after the toxin containing pharmaceutical composition is formulated, the toxin is stabilized with a stabilizing agent or excipient. Acceptable excipients or stabilizers include protein excipients, such as albumin or gelatin, or the like, or non-protein excipients, including poloxamers, saccharides, polyethylene glycol, hyaluronic acid or the like. Use of non-protein excipients in botulinum toxin formulations is disclosed in U.S. Pat. Nos. 10,360,190; 10,973,890; PCT publications and WO2018053021; WO2018053004 and WO2020056371; each of which is hereby incorporated by reference in its entirety.

Animal protein free and/or chromatographic methods for obtaining a botulinum neurotoxin are disclosed in U.S. Pat. Nos. 7,160,699; 7,354,740; 7,189,541; 7,445,914; 7,452,697; 7,560,251; 8,409,828; 8,008,044; 8,012,716; 8,841,110 and 9,725,705 and 7,189,541; each of which is hereby incorporated by reference in its entirety. Animal protein free processes and systems for obtaining a botulinum neurotoxin are also disclosed in U.S. Pat. Nos. 8,129,139; 8,927,229; 8,357,541; 8,932,827; 8,324,349; 9,206,409; 9,719,076; 10;465,178; 11,124,786; 11,203,748; 11,326,155 and U.S. patent application Ser. Nos. 17/579,208; 17/694,932; 17/694,962; 17/732,951; 17/835,503; 17/953,789 and 17/953,882; each of which is hereby incorporated by reference in its entirety.

It is contemplated that any of the currently available or future commercially available botulinum toxin formulations are suitable for the methods described herein, including but not limited to gemibotulinumtoxinA, daxibotulinumtoxinA, nivobotulinumtoxinA, BOTOX® (onabotulinumtoxinA), DYSPORT® (abobotulinumtoxinA), XEOMIN® (incobotulinumtoxinA), JEUVEAU™ (prabotulinumtoxinA), BOTULAX® (letibotulinumtoxinA), NEURONOX®, and INNOTOX® (nivobotulinumtoxinA).

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

In some embodiments, the botulinum toxin is selected from the group consisting of botulinum toxin types A, B, C, D, E, F and G. In one embodiment, the clostridial derivative of the present method is a botulinum toxin type A (or serotype A). The botulinum toxin can be a recombinant botulinum neurotoxin, such as botulinum toxins produced by E. coli.

In some embodiments, the botulinum toxin type A is selected from onabotulinumtoxinA, incobotulinumtoxinA, abotulinumtoxinA, daxibotulinumtoxinA, prabotulinumtoxinA, letibotulinumtoxinA, lanbotulinumtoxinA, nivobotulinumtoxinA, gemibotulinumtoxinA and NEURONOX®.

In some embodiments, the botulinum toxin is a pure neurotoxin, devoid of complexing proteins. In some embodiments, the pure neurotoxin is selected from incobotulinumtoxinA and daxibotulinumtoxinA.

In some embodiments, the botulinum toxin is in an animal protein free formulation. In some embodiments, the botulinum toxin is gemibotulinumtoxinA. In an alternative embodiment, the botulinum toxin is nivobotulinumtoxinA. In another embodiment, the botulinum toxin is daxibotulinumtoxinA.

The botulinum toxin for use according to the present methods can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. Acceptable excipients or stabilizers include protein excipients, such as albumin or gelatin, or the like, or non-protein excipients, including poloxamers, saccharides, polyethylene glycol, or the like. In embodiments containing albumin, the albumin can be, for example, human serum albumin or recombinant human albumin, or the like. The lyophilized material can be reconstituted with a suitable liquid such as, for example, saline, water, or the like to create a solution or composition containing the botulinum toxin to be administered to the subject.

In some embodiments, the botulinum toxin serotype A (e.g., gemibotulinumtoxinA) is provided in a lyophilized formulation comprising trehalose, poloxamer P188, L-methionine and histidine. In some embodiments, the botulinum toxin formulation comprises 2% trehalose, 4% poloxamer P188, 0.2% L-methionine and 20 mM Histidine. In some embodiments, the formulation further comprises NaCl. In some embodiment, the formulation comprises 0.6% NaCl.

In some embodiments, the botulinum toxin serotype A (e.g., gemibotulinumtoxinA) is provided in a solution. In some embodiments, the botulinum toxin formulation comprises 8% trehalose, 4% poloxamer P188, 0.2% L-methionine and 20 mM Histidine.

In some embodiments, to increase the resident time of the clostridial derivative at the site of injection, the clostridial derivative is provided in a controlled release system comprising a polymeric matrix encapsulating the clostridial derivative, wherein fractional amount of the clostridial derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety.

The effective amount of the clostridial derivative, for example a botulinum toxin, administered according to the present method can vary according to the potency of the toxin and particular characteristics of the condition being treated, including its severity and other various subject variables including size, weight, age, and responsiveness to therapy. The potency of the toxin is expressed as a multiple of the LD₅₀ value for the mouse, where one unit (U) of toxin may be defined as the equivalent amount of toxin that kills 50% of a group of 18 to 20 female Swiss-Webster mice, weighing about 20 grams each or through a cell-based potency assay such as described in U.S. 2010/0203559 and U.S. 2010/0233802.

The therapeutically effective amount of the botulinum toxin can vary according to the potency of a botulinum toxin, as commercially available botulinum toxin formulations do not have equivalent potency units.

In some embodiments, the effective amount of the botulinum toxin type A administered in accord with the method is between about 10-1000 units, 25-800 units, 25-750 units, 50-725 units, 75-500 units, 100-400 units, 120-350 units, or 100-200 units. In some embodiments, the effective amount is divided into equal or unequal portions, and a portion of between about 5-150 units, 10-125 units, 10-100 units, 10-80 units, 10-40 units, 15-100 units, 20-100 units, 20-80 units, 20-70 units, 20-60 units, 20-30 units, or 40-60 units is administered to each epicardial fat pad. In some embodiments, about 10 units, 15 units, 20 units, 25 units, 30 units, 35 units, 40 units, 45 units, 50 units, 55 units or 60 units is administered to each epicardial fat pad. In one embodiment, the portions administered to each epicardial fat pad are equal. In another embodiment, the portions administered to each epicardial fat pad are unequal, and in another embodiment, the portions administered to each epicardial fat pad are a combination of equal and unequal.

In some embodiments, the botulinum toxin is selected from the group consisting of botulinum toxin types A, B, C, D, E, F and G. In some embodiments, the botulinum toxin is a botulinum toxin type A (or serotype A). The botulinum toxin type A can be administered in accord with the treatment method to the epicardial fat pads to deliver to the subject a total dose comprising an effective amount of between about 1 unit and about 3,000 units, or between about 2 units and about 2000 units, or between about 5 units and about 1000 units, or between about 10 units and about 500 units, or between about 15 units and about 250 units, or between about 20 units and about 150 units, or between 25 units and about 100 units, or between about 30 units and about 75 units, or between about 35 units and about 50 units, or the like.

In some embodiments, the botulinum toxin serotype A is administered in accordance with the treatment method to the epicardial fat pads at a total dose of about 125 units of animal free botulinum toxin, e.g., at a dose of about 25 units to each of five epicardial fat pads. In some embodiments, the botulinum toxin serotype A is administered in accord with the treatment method to the epicardial fat pads at a total dose of about 250 units of animal free botulinum toxin, e.g., at a dose of about 50 units to each of five epicardial fat pads. In some embodiments, the botulinum toxin serotype A is gemibotulinumtoxinA. In some embodiments, the botulinum toxin serotype A is daxibotulinumtoxinA. In some embodiments, the botulinum toxin serotype A is nivobotulinumtoxinA.

In some embodiments, the botulinum toxin serotype A is administered at about 10 units to about 40 units to each of the five epicardial fat pads for a total dose of about 125 units. In some embodiments, the animal free botulinum toxin is administered at about 20 units to about 30 units to each of the five epicardial fat pads for a total dose of about 125 units. In some embodiments, the animal free botulinum toxin is administered at about 25 units to each of the five epicardial fat pads for a total dose of about 125 units.

In some embodiments, the administration of the botulinum toxin serotype A according the methods described herein comprises injecting 1 mL of a botulinum toxin serotype A solution having a concentration of 25 units/mL into each of the five epicardial fat pads. In some other embodiments, the methods comprise injecting 0.5 mL of a botulinum toxin serotype A solution having a concentration of 50 units/mL into each of the five epicardial fat pads.

In some embodiments, the botulinum toxin serotype A is administered at about 25 units to about 75 units to each of the five epicardial fat pads for a total dose of about 250 units. In some embodiments, the animal free botulinum toxin is administered at about 40 units to about 60 units to each of the five epicardial fat pads for a total dose of about 250 units. In some embodiments, the animal free botulinum toxin is administered at about 50 units to each of the five epicardial fat pads for a total dose of about 250 units.

The amount (dose) of toxin administered and/or the frequency of its administration will be at the discretion of the physician responsible for the treatment and will be commensurate with questions of safety and the effects produced by a particular toxin formulation. In one embodiment, treatment effects of the botulinum toxin persist for between about 1 month and 5 years, or for between about 1 month and 1 year, or for between about 1 month and 6 months, or for between about 1 month and 5 months, for between about 1 month and 4 months, for between about 1 month and 3 months, for between about 1 month and 2 months. In one embodiment, the effect of the botulinum toxin persists for a period of time sufficient to prevent POAF and/or attenuate risk of POAF.

In certain embodiments, the dose of a botulinum toxin used according to embodiments of the present method is less than the amount of botulinum toxin that would be used to paralyze a muscle. In one embodiment, the effective about of botulinum toxin type A administered in accord with the method is 250 units or less.

In one embodiment, the effective amount of botulinum toxin administered is in the form of a composition that can be a stable liquid or solid (e.g., lyophilized) pharmaceutical composition. The composition may comprise the clostridial toxin and a pharmacologically acceptable excipient. As used herein “pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient or permitted to dilute or enclose the active ingredient and can be a solid, semi-solid, or liquid agent. In an embodiment, the composition comprises the botulinum toxin, a disaccharide, a surfactant and an antioxidant. In another embodiment, the composition comprises the botulinum toxin, a disaccharide, a surfactant and an animal protein, such as an albumin. In another embodiment, the composition comprises the botulinum toxin, a disaccharide, a surfactant and does not contain an animal protein; that is, the composition is an animal protein free composition.

It is also envisioned that a pharmaceutical composition comprising a Clostridial toxin active ingredient can include one or more pharmaceutically acceptable excipients that facilitate processing of an active ingredient into pharmaceutically acceptable compositions. Insofar as any pharmacologically acceptable excipient is not incompatible with the Clostridial toxin active ingredient, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003), each of which is hereby incorporated by reference in its entirety.

The constituent ingredients of a pharmaceutical composition can be included in a single composition (that is, all the constituent ingredients, except for any required reconstitution fluid, are present at the time of initial compounding of the pharmaceutical composition) or as a two-component system, for example a vacuum-dried composition reconstituted with a reconstitution vehicle which can, for example, contain an ingredient not present in the initial compounding of the pharmaceutical composition. A two-component system can provide several benefits, including that of allowing incorporation of ingredients which are not sufficiently compatible for long-term shelf storage with the first component of the two-component system. For example, the reconstitution vehicle may include a preservative which provides sufficient protection against microbial growth for the use period, for example one-week of refrigerated storage, but is not present during the two-year freezer storage period during which time it might degrade the toxin. Other ingredients, which may not be compatible with a botulinum toxin or other ingredients for long periods of time, can be incorporated in this manner; that is, added in a second vehicle (e.g., in the reconstitution vehicle) at the approximate time of use.

The pharmaceutical composition can also include preservative agents such as benzyl alcohol, benzoic acid, phenol, parabens and sorbic acid. Pharmaceutical compositions can include, for example, excipients, such as surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

In one embodiment, the botulinum toxin is provided in single use vials in the form of lyophilized powder. For example, the vial contains, in some embodiments, between about 10-750 units or between about 100-240 units of a botulinum toxin type A, and any excipients. Prior to use, the lyophilate can be reconstituted with, for example, sterile, non-preserved 0.9% Sodium Chloride Injection USP. In one embodiment, a vial comprises lyophilized 50 units or 200 units of botulinum toxin for reconstitution with sterile, non-preserved 0.9% sodium chloride USP.

In other embodiments, the botulinum toxin is provided in single-use pre-filled syringes. Pre-filled syringes are pre-reconstituted with sterile, non-preserved 0.9% sodium chloride Injection USP. The pre-filled syringes can comprise between about 1-5 mL, or preferably about 3 mL, and may comprise between about 10-150 units, or between about 20-100 units, 20-60 units, or 25-50 units of a botulinum toxin type A. In one embodiment, a pre-filled syringe comprises 3 mL with 50 units or 200 units of a botulinum toxin type A.

### III. EXAMPLES

The following examples are illustrative in nature and are in no way intended to be limiting.

### Example 1: A Phase 2 Clinical Study on Injection of Botulinum Toxin

### Into Epicardial Fat Pads

A multi-center, Phase 2 randomized, double-blind, placebo-controlled, parallel group dose-ranging study was carried out to evaluate the efficacy and safety of botulinum toxin type A injection into the epicardial fat pads to prevent POAF in patients undergoing cardiac surgery.

### Demographics, Baseline Characteristics and Disposition of Patients

The study enrolled adult patients ages of 55-90, inclusive, who were scheduled to undergo cardiac surgery, including open-chest coronary artery bypass grafting and/or valve surgery. Patients younger than 55 were not eligible. Table 1 below lists the principal inclusion and exclusion criteria for the study.

TABLE 1
Principal inclusion and exclusion criteria for the study

| Inclusion Criteria | Exclusion Criteria |
|---|---|
| Age 55-90 years of age, inclusive | Any medical condition that places the |
| Scheduled to undergo open-chest cardiac | participant at an increased risk with |
| surgery | exposure to botulinum toxin type A, |
| Including coronary artery bypass grafting | including diagnosed muscular dystrophy, |
| (CABG) and/or valve repair/replacement | myasthenia gravis, Eaton-Lambert |
| If male, willingness to minimize the risk of | syndrome, amyotrophic lateral sclerosis, |
| inducing pregnancy for 60 days. If female, | mitochondrial disease, or any other |
| not pregnant, not breast-feeding, and willing | significant disease which might interfere |
| to follow contraceptive guidance. | with neuromuscular function. |
| In sinus rhythm in the 48 hours before | Aspiration pneumonia, lower respiratory |
| surgery. | tract infections, uncontrolled asthma, severe |
| Willing to wear an electrocardiogram (ECG) | chronic obstructive pulmonary disease, or |
| patch for 30 days post-surgery and for 7 days | otherwise compromised respiratory function |
| after each study visit | within the past 3 months. |
| | Permanent or persistent atrial fibrillation. |
| | Allergy or sensitivity to botulinum toxin |
| | type A or medical adhesive. |
| | Severe left atrial enlargement (left atrial |
| | diameter >55 mm). |
| | Left ventricular ejection fraction <25%. |
| | Presence or history of symptomatic 2^{nd} |
| | degree or 3^{rd} degree atrioventricular block in |
| | the last 30 days. |
| | Class I or class III antiarrhythmic drug |
| | therapy. |
| | Botulinum toxin type A use within 6 months. |
| | Preoperative need to inotropes, |
| | vasopressors, or intra-aortic balloon counter |
| | pulsation. |
| | Prior cardiac surgery. |
| | History of AF ablation or planned ablation at |
| | the time of surgery. |
| | Emergency cardiac surgery. |
| | Concurrent enrollment in an investigational |
| | study drug or device study within 30 days of |
| | study entry. |
| | Impaired prognosis defined as EuroSCORE |
| | II greater than 7% perioperative mortality. |
| | Females who are pregnant, nursing, or |
| | planning pregnancy. |

Patients were divided into three interventional arms randomized in a 1:1:1 fashion: 125 units of a botulinum toxin type A in an animal protein free formulation (such as gemibotulinumtoxinA) (25 units per fat pad), 250 units of botulinum toxin type A (50 units per fat pad), and placebo (see Table 2). Randomization was stratified by age (<65, ≥65 years) and the type of surgery (Valve/combo surgery or isolated CABG surgery), and patient characteristics were generally balanced between treatment arms (see Table 3).

TABLE 2
Disposition of patients enrolled in the study

| Subject | Placebo | | 125 U | | 250 U | | Total | |
|---|---|---|---|---|---|---|---|---|
| Disposition | n (%) | | n (%) | | n (%) | | n (%) | |
| Randomized | 108 | (100) | 106 | (100) | 109 | (100) | 323 | (100) |
| Treated | 105 | (97.2) | 105 | (99.1) | 109 | (100) | 319 | (98.8) |
| Discontinued Study | 13 | (12.0) | 13 | (12.3) | 13 | (11.9) | 39 | (12.1) |
| (Primary Reason) | | | | | | | | |
| Adverse Event | 1 | (0.9) | 0 | | 0 | | 1 | (0.3) |
| Withdrawal by Subject | 4 | (3.7) | 6 | (5.7) | 5 | (4.6) | 15 | (4.6) |
| Lost to Follow-Up | 5 | (4.6) | 6 | (5.7) | 6 | (5.5) | 17 | (5.3) |

| | | | | | | |
|---|---|---|---|---|---|---|
| Death | 0 | 0 | 2 | (1.8) | 2 | (0.6) |

| | | | | | | | |
|---|---|---|---|---|---|---|---|
| Physician Decision | 1 | (0.9) | 1 | (0.9) | 0 | 2 | (0.6) |

| | | | | | | |
|---|---|---|---|---|---|---|
| Protocol Deviation | 1 | (0.9) | 0 | 0 | 1 | (0.3) |
| Other: Medic. Monitor Decision | 1 | (0.9) | 0 | 0 | 1 | (0.3) |

TABLE 3
Demographics and baseline characteristics of treated patients

| Demographics and | | | | | | |
|---|---|---|---|---|---|---|
| Baseline Characteristics | Placebo | | 125 U | | 250 U | |
| Mean (SD) or n (%) | (N = 105) | | (N = 105) | | (N = 109) | |
| Male | 91 | (86.7) | 88 | (83.8) | 87 | (79.8) |
| Age (years) | | | | | | |
| Mean (Range) | 66.8 | (55, 80) | 66.8 | (55, 84) | 67.2 | (56, 84) |
| <65, n % | 43 | (41.0) | 42 | (40.0) | 45 | (41.3) |
| ≥65, n % | 62 | (59.0) | 63 | (60.0) | 64 | (58.7) |
| Type of Surgery (Actual) | | | | | | |
| CABG* | 65 | (61.9) | 68 | (64.8) | 70 | (64.2) |
| Valve | 27 | (25.7) | 24 | (22.9) | 27 | (24.8) |
| CABG and Valve | 13 | (12.4) | 13 | (12.4) | 12 | (11.0) |
| Stratification Groups | | | | | | |
| Valve Surgery, Age <65 years | 17 | (16.2) | 14 | (13.3) | 15 | (13.8) |
| Valve Surgery, Age ≥65 years | 23 | (21.9) | 23 | (21.9) | 24 | (22.0) |
| No Valve Surgery, Age <65 years | 26 | (24.8) | 28 | (26.7) | 30 | (27.5) |
| No Valve Surgery, Age ≥65 years | 39 | (37.1) | 40 | (38.1) | 40 | (36.7) |

*CABG: coronary artery bypass grafting.

### Treatment and Assessment of Treatment Efficacy

Either placebo or botulinum toxin type A (at one of two dosages, 125U or 250U) was injected in the five epicardial fat pads (4 associated with the pulmonary veins as well as the aortic-pulmonary vein fat pad) during the open-chest CABG surgery. The injection pattern or manner was within the scope of the methods disclosed herein. Intraoperative, concomitant ablation was not permitted. Consistent with current guidelines, perioperative beta-blocker therapy was recommended if the investigator deemed it appropriate.

The injection paradigm used in the clinical trial included, for example, administering the botulinum type A to the fat pads in a specific sequence, such as performing the injections of botulinum toxin type A into the aortic fat pad first followed by the two right side pulmonary vein fat pads then the two left side pulmonary vein fat pads or followed by the two left side pulmonary vein fat pads then the two right side pulmonary vein fat pads; performing the injections of botulinum toxin type A into the five fat pads; administering the botulinum toxin before the primary surgery including prior to initiation of cardiopulmonary bypass, during bypass cannula placement, during cardiopulmonary bypass, and/or after cardiopulmonary bypass; minimizing the number of punctures per fat pad to not more than three (such as 1 to 3 injections to aortic fat pad; single injection to each of the right and left side pulmonary vein fat pads); and bending the needle used in the injection to most readily achieve efficient angle. Injections made at before the primary surgery avoided disturbing any of the coronary artery bypass grafting (CABG) and/or valve work. Other advantages included for example, reducing injection time, bypass time, and chance for leakage and tissue trauma, increasing safety for patient and reducing risks for complications.

The study was divided into a screening period, intraoperative treatment, and the follow-up period. The follow-up period included the first 30 days and up to one-year after surgery. The primary and secondary efficacy assessments were performed through Day 30. All patients were followed out to at least Day 367 for additional and safety assessments.

Primary and secondary efficacy endpoints were assessed using continuous ECG monitoring for 30 days following cardiac surgery. Primary endpoint included percentage of patients with continuous AF≥30 seconds (sec). Secondary endpoints included several measures of AF frequency, duration, and burden. Additional endpoints included clinically important tachycardia in AF, time to AF termination, and healthcare utilization. All patients were followed for up to 1 year for safety.

### Injection Procedures and Technique

The following instructions were provided to the surgeons to perform toxin injections for the clinical study described herein:
On-pump procedures are recommended, as the heart often needs to be lifted or shifted or moved to gain access to the posterior fat pads.
Injections should be made at the beginning of the procedure, to avoid disturbing any of the CABG and/or valve work.
If the heart is to be arrested, the injections can be performed after the induction of cardiac arrest.
Retain the anterior fat pad when clamping, if possible.
If a peri-PV fat pad requires transection for performance of the surgical procedure, e.g., mitral valve case, this should be undertaken as per customary approach, and this should be documented.
Care should be taken not to inject study drug into the coronary arteries or coronary sinus when injecting fat pads on the left side. Similarly, given the proximity of the left and right main coronary to the anterior fat pad, injections should be kept superficial, and toxin should not be injected into the coronary arteries.
Inclusionary valve repair/replacement procedures for the primary reason for surgery include:
   Aortic valve repair/replacement
   Mitral valve repair/replacement
   Combination of Aortic and Tricuspid valve repair/replacement
   Combination of Mitral and Tricuspid valve repair/replacement
   CABG/Valve combination procedures (when valvular procedure is one of 4 sub-bulleted ones immediately above)
   Left Atrial Appendage (LAA) exclusions are allowed, however are not a qualifying surgical procedure on their own
Exclusionary valve repair/replacement procedures included but were not limited to:
   Combination of Aortic and Mitral valve repair/replacement
   Isolated tricuspid valve repair/replacement

Upon completion of the sternotomy to expose the heart in preparation of the CABG Procedure the epicardial fat pads can be located for injection. The following should be anticipated during the surgical procedures and injection technique:
Each fat pad should be injected with 1 mL of study drug, and it is recommended that each mL is distributed across 1-3 injections per fat pad.
Anticipate 5-15 seconds per injection, or 10-45 seconds total per fat pad
Injections should be made at a depth of 1-2 mm and at an oblique angle
Visible swelling of the fat pad and the appearance of a wheal should be expected upon injection of Study Intervention

### Injection of Aortic Fat Pad

Little to no manipulation of the heart is needed to gain access to the aortic fat pad. A syringe with a 25 G to 30 G needle is used to inject the total volume of 1 mL, this should be injected into 1 to 3 different regions of the fat pad. Swelling of the fat pad is expected with little to no fluid leakage. If leakage occurs, this should be documented. Significant leakage, defined as greater than 50% of intended volume for that target, must be documented as a not successful injection.

### Injection of Left Pulmonary Vein Fat Pads

The heart must be shifted to the right to expose the left superior fat pad and the left inferior fat pad. Additionally, the left atrial appendage often must be retracted to access the left superior fat pad, and the apex of the heart may require retraction to access the left inferior fat pad. Syringes with a 25 G to 30 G needle are used to inject the total volume of 1 mL into each fat pad, this should be performed with a single injection into each fat pad. Swelling of the fat pads is expected with little to no fluid leakage. If leakage occurs, this should be documented. Significant leakage, defined as greater than 50% of intended volume for that target, must be documented as a not successful injection.

### Injection of Right Pulmonary Vein Fat Pad

The heart must be shifted to the left to expose the right superior fat pad and the right inferior fat pad. Syringes with a 25 G to 30 G needle are used to inject the total volume of 1 mL into each fat pad, this should be performed with a single injection into each fat pad. Swelling of the fat pads is expected with little to no fluid leakage. If leakage occurs, this should be documented. Significant leakage, defined as greater than 50% of intended volume for that target, must be documented as a not successful injection.

### Recording of Injection Procedure and Fat Pad Morphology

Data on the injection procedure and Pericardial or epicardial morphology noted for each of the five fat pads must be recorded in the eCRF. It is recommended that this be collected concurrently with the procedure.

Details should be collected regarding the injection procedure to answer the following questions for reporting in the eCRF. 1) was the injection site successfully identified, 2) was the epicardial fat pad injected, 3) what was the order of the fat pad injection (e.g. which was injected first, second, third, etc.), 4) was the fat pad transected or dissected during the procedure, 5) was there any swelling (i.e. an appearance of a wheal) upon injection, 6) was there any significant leakage (any leakage greater than 50% of intended volume for that target is considered significant and should be documented) from the fat pad following injection, and 7) were all contents of the syringe successfully injected (significant leakage, defined as greater than 50% of intended volume for that target, must be documented as a not successful injection), and if not, did A) all or most of the study drug leak out of the fat pad, B) was all or most of the study medication injected into the incorrect structure, or C) all or most of the study medication could not be injected due to other reasons (e.g., needle/syringe malfunction), 8) segment of procedure when the injections were performed (e.g., on-bypass, prior to cross-clamp; on-bypass, post cross-clamp; on by-pass, post-procedure; off-bypass).

Should fat pad abnormalities found it should be noted in the eCRF if the fat pad was not found, the fat pad was not accessible, the fat pad had a markedly abnormal shape, the fat pad was in an atypical location, or if none of these apply then the abnormality should be specified.

### Completion of Injections and Surgical Procedures

Upon completion of injections into all 5 epicardial fat pads, the CABG and/or valve replacement should commence per standard local practice guidelines and regulations. Upon completion of the surgery the study participant should have an ECG ePatch applied.

### Example 2: Efficacy of Toxin Treatments Across all Patients

The primary endpoint was assessed for 312 participants who made up the modified intention-to-treat (mITT) population. The mITT comprised participants who were treated with the study intervention of botulinum toxin type A (gemibotulinumtoxinA) and had at least one post-dose electrocardiogram (ECG) by Day 30. The primary endpoint of an AF episode≥30 sec during the first 30 days post-surgery was not statistically different across the three treatment groups. AF episodes≥30 sec occurred in 47 patients (46.1%) of the placebo group, 38 patients (36.5%) of the 125 U group (RR vs placebo, 0.80 [95% CI 0.58, 1.10], P=0.16) and 50 patients (47.2%) of the 250 U group (RR vs placebo, 1.04 [95% CI 0.79, 1.37], P=0.78). Overall, there were numerical reductions in the rates of POAF in the 125U treatment group compared to placebo.

Among CABG and/or valve surgery participants who had a beta blocking agent withdrawn within 3 days post-surgery and no replacement within 7 days post-surgery (n=196, 61%), the primary endpoint (AF≥30 sec) occurred in 18 (31.6%) of the 125U group compared with 31 (46.3%) of the placebo group (RR 0.66 [95% CI 0.42-1.05]P=0.0597).

Additionally, different durations of POAF were tested across the three treatment groups. These secondary endpoints did not meet the nominal level at which they were tested (p<0.05). It should be noted that all p values, other than the primary endpoint, were not controlled for multiplicity and as such are nominal p values. Overall, the 125U treatment group demonstrated consistent numerical reductions in the rate of POAF compared to placebo.

Regarding safety, the rate of adverse events (AE) and severe adverse events (SAE) observed among patients receiving the 125U dose was comparable to the rate among patients receiving the placebo treatment. The 250U dose resulted in a rate of AE that was numerically higher that placebo group, but SAE rate was not higher than placebo. There were no increases observed for potential distant spread of toxin, and no meaningful differences in pulmonary function tests. Therefore, overall, the data raised no particular safety concern about the studied toxin treatments.

### Example 3: Significant Efficacy of Toxin Treatments Observed Among Isolated CABG Patients

Analysis of treatment efficacy among a subgroup of patients who received valve surgery with CABG shows no significant benefits in injecting the toxin at either dose (data not shown). However, among participants undergoing isolated CABG (without valve surgery) (n=199, 64%), significant improvements were observed in the toxin treated groups compared to the placebo group.

Specifically, the primary endpoint (AF≥30 see) occurred in 19 patients (28.4%) of the 125 U group compared with 26 patients (40%) in the placebo group (RR 0.71, [95% CI 0.44, 1.15], P=0.16) and 23 patients (34.3%) of the 250 U group compared with 26 patients (40%) in the placebo group (RR 0.89, [95% CI 0.58-1.37], P=0.60). Furthermore, the secondary endpoint of AF≥4 hours occurred in 11 patients (16.4%) of the 125U group compared with 20 patients (30.8%) in the placebo group (RR 0.53, [95% CI 0.28-1.02]P=0.0497) thus achieving a nominal p value<0.05. A summary of the data can be found in Table 4 below.

TABLE 4
Analysis of no-valve surgery subgroup of patients

| | Placebo | 125 U | 250 U |
|---|---|---|---|
| | (N = 65) | (N = 67) | (N = 67) |
| PRIMARY ENDPOINT | | | |
| AF Episode ≥30 seconds, n (%) | 26 (40.0) | 19 (28.4) | 23 (34.3) |
| Odds Ratio vs. Placebo (95% CI) | | 0.60 (0.29, 1.23) | 0.82 (0.39, 1.72) |
| Relative Risk vs. Placebo (95% CI) | | 0.71 (0.44, 1.15) | 0.89 (0.58, 1.37) |
| P-value | | 0.1552 | 0.5998 |
| SECONDARY ENDPOINT | | | |
| (Alternative Duration) | | | |
| AF Episode ≥5 minutes, n (%) | 25 (38.5) | 17 (25.4) | 22 (32.8) |
| Odds Ratio vs. Placebo (95% CI) | | 0.55 (0.26, 1.15) | 0.82 (0.39, 1.71) |
| Relative Risk vs. Placebo (95% CI) | | 0.66 (0.39, 1.11) | 0.89 (0.57, 1.39) |
| P-value | | 0.1038 | 0.5926 |
| AF Episode ≥4 hours, n (%) | 20 (30.8) | 11 (16.4) | 11 (16.4) |
| Odds Ratio vs. Placebo (95% CI) | | 0.44 (0.19, 1.01) | 0.45 (0.19, 1.05) |
| Relative Risk vs. Placebo (95% CI) | | 0.53 (0.28, 1.02) | 0.55 (0.29, 1.06) |
| P-value | | 0.0497 | 0.0635 |

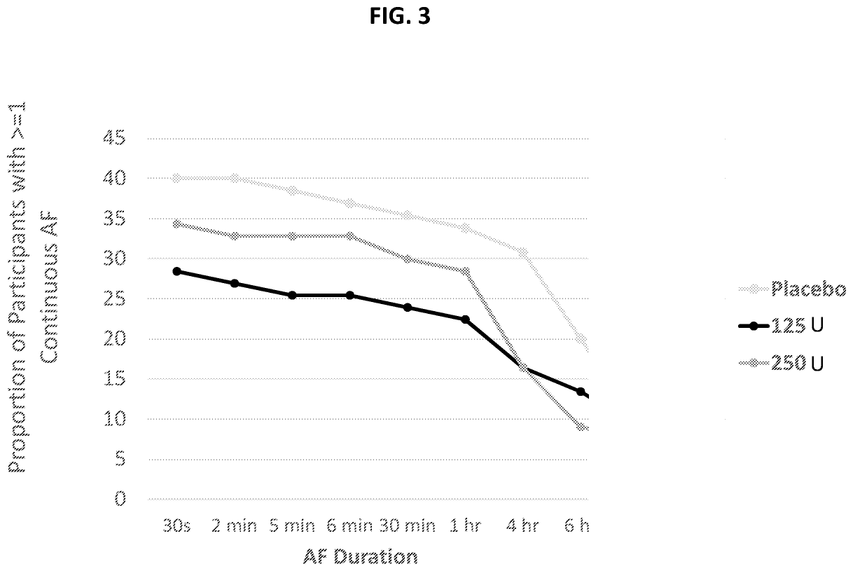

It can be seen from FIG. 3 and the relative risk (RR) reduction data shown in Table 5 that treatment with 125U and 250U toxin reduced the rate of occurrence of POAF episodes having a duration equal to or greater than 30 sec and occurrence of POAF episodes having a duration equal to or greater than 4 hours. The 125U dose appeared to perform better than the 250U dose for AF episodes≥1 hour or less, but both doses remarkably reduced the risk of AF episodes≥4 hours by about 45%. Reduction of AF episodes≥4 hours by the 125U dose also achieved statistical significance compared to the placebo group.

TABLE 5
Relative risk reduction rate by 125 U and 250 U toxin
treatment among no-valve surgery subgroup of patients

| | | Relative Risk Reduction | Relative Risk Reduction |
|---|---|---|---|
| AF Duration | | (125 U) | (250 U) |
| >=30 | seconds | 29% | 11% |
| >=5 | minutes | 34% | 11% |
| >=4 | hours | 47%* | 45% |

*p < 0.05 compared with placebo

Among participants who had a beta blocking agent withdrawn and underwent an isolated CABG without valve surgery (n=118, 37%), the primary endpoint (AF≥30 sec) occurred in 6 (17.6%) of the 125U group compared with 16 (38.1%) of the placebo group (RR 0.48 [95% CI 0.21-1.08]P=0.0464). Beta blocker therapy is standard of care for patients getting cardiac surgery, especially CABG patients having lower heart rates and blood pressures. After cardiac surgery, for some patients, beta blocker therapy is withdrawn because of various medical reasons, including for example high blood loss, or low heart rates. The withdrawal of beta blocker therapy increases the incidences of post-operative atrial fibrillation in these patients. The data presented herein shows that administration of 125U of gemibotulinumtoxinA reduced the risk of AF episodes≥30 sec by about 48% and this reduction also achieved statistical significance compared to the placebo group.

It is clear from the data presented herein that treatment with botulinum toxin serotype A (e.g., gemibotulinumtoxinA), particularly with the 125U dose, remarkably reduced POAF rate for patients receiving CABG without valve surgery. Therefore, CABG patients who are not receiving valve surgery can benefit greatly from toxin administration as described in the present methods before or during CABG surgery.

### Example 4: Significant Efficacy of Toxin Treatments Observed Among Patients of 65 Years of Age and Older

Analysis of treatment efficacy among a subgroup of patients of less than 65 years old shows no significant benefits in injecting toxin at either dose (data not shown). Among participants of at least 65 years old (n=185), significant improvements were observed, particularly in the 125U treatment group, compared to the placebo group.

Specifically, the primary endpoint (AF≥30 see) occurred in 23 patients (36.5%) of the 125 U group compared with 35 patients (57.4%) in the placebo group (RR 0.64, [95% CI 0.43, 0.94], P=0.0194), achieving a nominal p value<0.05. Furthermore, the secondary endpoint of AF>5 minutes occurred in 22 patients (34.9%) of the 125U group compared with 35 patients (54.1%) in the placebo group (RR 0.64, [95% CI 0.43-0.97]P=0.0309), also achieving statistical significance. A summary of the data can be found in Table 6 below.

TABLE 6
Analysis of subgroup of patients ≥65 years old

| | Placebo | 125 U | 250 U |
|---|---|---|---|
| | (N = 61) | (N = 63) | (N = 61) |
| PRIMARY ENDPOINT | | | |
| AF Episode ≥30 seconds, n (%) | 35 (57.4) | 23 (36.5) | 33 (54.1) |
| Odds Ratio vs. Placebo (95% CI) | | 0.42 (0.21, 0.88) | 0.86 (0.42, 1.76) |
| Relative Risk vs. Placebo (95% CI) | | 0.64 (0.43, 0.94) | 0.93 (0.68, 1.28) |
| P-value | | 0.0194 | 0.6734 |
| SECONDARY ENDPOINT | | | |
| (Alternative Duration) | | | |
| AF Episode ≥5 minutes, n (%) | 33 (54.1) | 22 (34.9) | 32 (52.5) |
| Odds Ratio vs. Placebo (95% CI) | | 0.45 (0.22, 0.94) | 0.92 (0.45, 1.87) |
| Relative Risk vs. Placebo (95% CI) | | 0.64 (0.43, 0.97) | 0.96 (0.69, 1.34) |
| P-value | | 0.0309 | 0.8109 |
| AF Episode ≥4 hours, n (%) | 26 (42.6) | 18 (28.6) | 16 (26.2) |
| Odds Ratio vs. Placebo (95% CI) | | 0.54 (0.26, 1.14) | 0.47 (0.22, 1.02) |
| Relative Risk vs. Placebo (95% CI) | | 0.67 (0.41, 1.09) | 0.61 (0.36, 1.02) |
| P-value | | 0.0999 | 0.0542 |

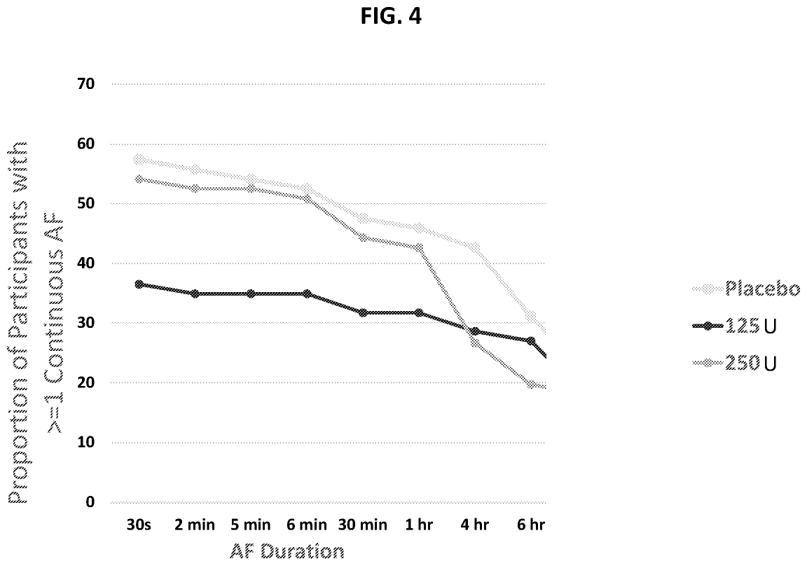

It can also be seen from FIG. 4 and the relative risk (RR) reduction data shown in Table 7 that treatment with 125U toxin generally lowered the rate of occurrence of POAF episodes having duration equal to or greater than 30 sec and the occurrence of POAF episodes having duration equal to or greater than 4 hours. In addition, the 125U dose surprisingly reduced AF episodes≥30 sec and ≥5 minutes with statistical significance, and both the 125U and 250U doses remarkably reduced the risk of AF episodes≥4 hours by at least 33% compared to placebo group.

TABLE 7
Relative risk reduction rate by 125 U and 250
U toxin treatment among ≥65 year-old patients

| | | Relative Risk Reduction | Relative Risk Reduction |
|---|---|---|---|
| AF Duration | | (125 U) | (250 U) |
| ≥30 | seconds | 36%* | 7% |
| ≥5 | minutes | 36%* | 4% |
| ≥4 | hours | 33% | 39% |

*p < 0.05 compared with placebo

It is clear from the data presented herein that treatment with toxin, particularly with the 125U dose, significantly reduced POAF occurrence rate for patients of 65 of age and older. Therefore, patients of about 65 years old or more can benefit greatly from toxin administration as disclosed in the present methods before or during open-chest cardiac surgery.

### Example 5: Significant Efficacy of Toxin Treatments Observed Among Isolated CABG Patients of 65 and Older

Examples 3 and 4 have surprisingly demonstrated that the subgroups of patients receiving isolated CABG (no-valve) surgery or patients of 65 years of age and older benefited from toxin injection during surgery. The effectiveness of toxin injection compared to placebo was even more pronounced among subgroup of patients who were both ≥65 years and did not receive valve surgery.

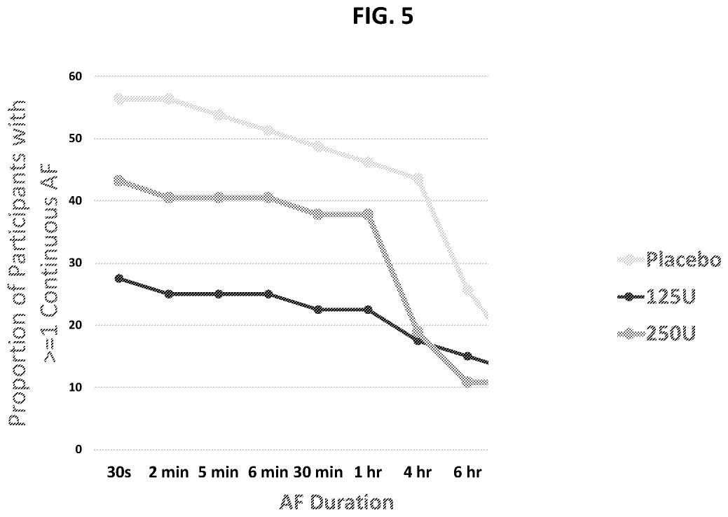

As depicted in FIG. 5 and the data in Table 8, injection of 125U of toxin significantly reduced occurrence of AF episodes≥30 sec, ≥5 min and ≥4 hours (all p values<0.05). The 250U injection also achieved significant reduction of AF episodes≥4 hours compared to placebo (p=0.0216).

TABLE 8
Analysis of subgroup of patients ≥65
years old and had CABG without valve surgery

| | Placebo | 125 U | 250 U |
|---|---|---|---|
| | (N = 39) | (N = 40) | (N = 37) |
| PRIMARY ENDPOINT | | | |
| AF Episode ≥30 seconds, n (%) | 22 (56.4) | 11 (27.5) | 16 (43.2) |
| Odds Ratio vs. Placebo (95% CI) | | 0.29 (0.11, 0.75) | 0.59 (0.24, 1.46) |
| Relative Risk vs. Placebo (95% CI) | | 0.49 (0.27, 0.87) | 0.77 (0.48, 1.22) |
| P-value | | 0.0096 | 0.2543 |
| SECONDARY ENDPOINT | | | |
| (Alternative Duration) | | | |
| AF Episode ≥5 minutes, n (%) | 21 (53.8) | 10 (25.0) | 15 (40.5) |
| Odds Ratio vs. Placebo (95% CI) | | 0.29 (0.11, 0.74) | 0.58 (0.24, 1.45) |
| Relative Risk vs. Placebo (95% CI) | | 0.46 (0.25, 0.85) | 0.75 (0.46, 1.22) |
| P-value | | 0.0091 | 0.2487 |
| AF Episode ≥4 hours, n (%) | 17 (43.6) | 7 (17.5) | 7 (18.9) |
| Odds Ratio vs. Placebo (95% CI) | | 0.27 (0.10, 0.77) | 0.30 (0.11, 0.85) |
| Relative Risk vs. Placebo (95% CI) | | 0.40 (0.19, 0.86) | 0.43 (0.20, 0.92) |
| P-value | | 0.0122 | 0.0216 |

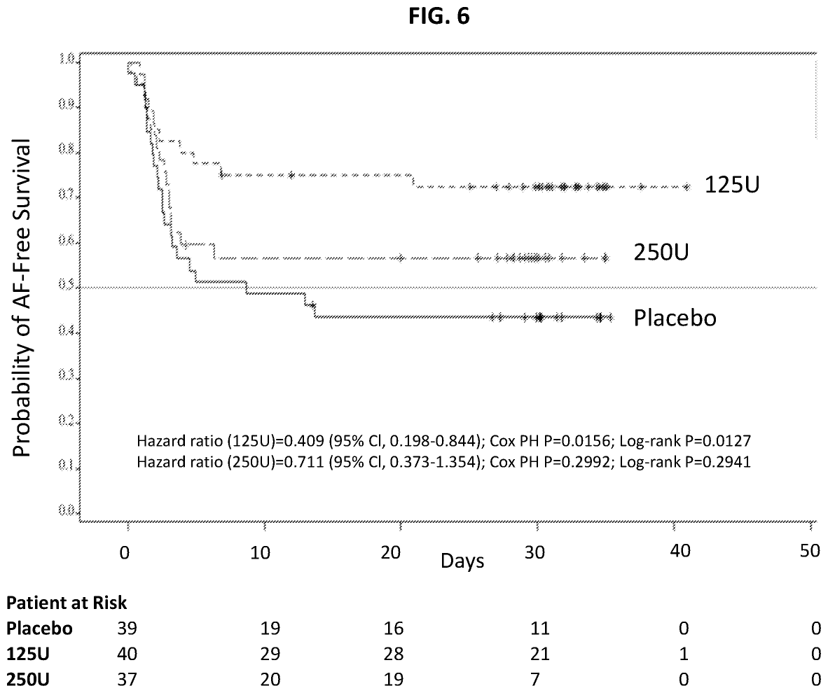

The effectiveness of toxin treatment of patients receiving CABG without valve surgery of 65 years of age and older can be further appreciated via the remarkable relative reduction risk, shown in Table 9. The 125U dose surprisingly reduced the risk of AF by 50% or more for both AF episodes≥30 sec, ≥5 min and ≥4 hours. The occurrence rate of AF episodes≥4 hours was also reduced by more than 500 with the 250U dose (Table 9). Furthermore, the probability of patients without AF (AF-free) during the first 30 days post-surgery was surprisingly better among patients receiving toxin injection, particularly the 125U dose, compared to patients receiving the placebo (see FIG. 6).

TABLE 9
Relative risk reduction rate by 125 U and 250 U toxin
treatment among isolated CABG and ≥65 year-old patients

| | | Relative Risk Reduction | Relative Risk Reduction |
|---|---|---|---|
| AF Duration | | (125 U) | (250 U) |
| >=30 | seconds | 51%** | 23% |
| >=5 | minutes | 54%** | 25% |
| >=4 | hours | 60%* | 57%* |

*p < 0.05 compared with placebo
**p < 0.01 compared with placebo

It is evident from these results that patients receiving CABG without valve surgery of 65 years of age or older can benefit greatly from toxin administration as disclosed in the present methods before or during open-chest CABG surgery.

### Example 6: Toxin Injection During Surgery Reduced Post-Surgery Clinical Burden

Exploratory endpoints included a variety of assessments of post-operative AF clinical burden, such as proportion of participants with at least one re-hospitalization post-discharge (all-cause and cardiovascular-related), time to re-hospitalization during first 30 days post-discharge and 60 days following surgery, hospital and ICU lengths of stay, hospital stay by AF status, and time to interventions due to AF including anticoagulants, antiarrhythmics, and procedural interventions.

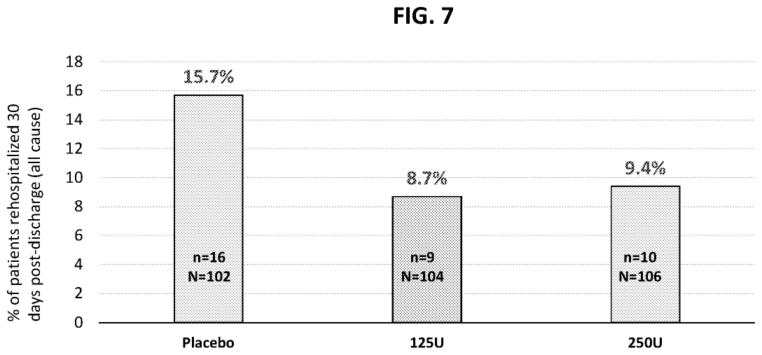

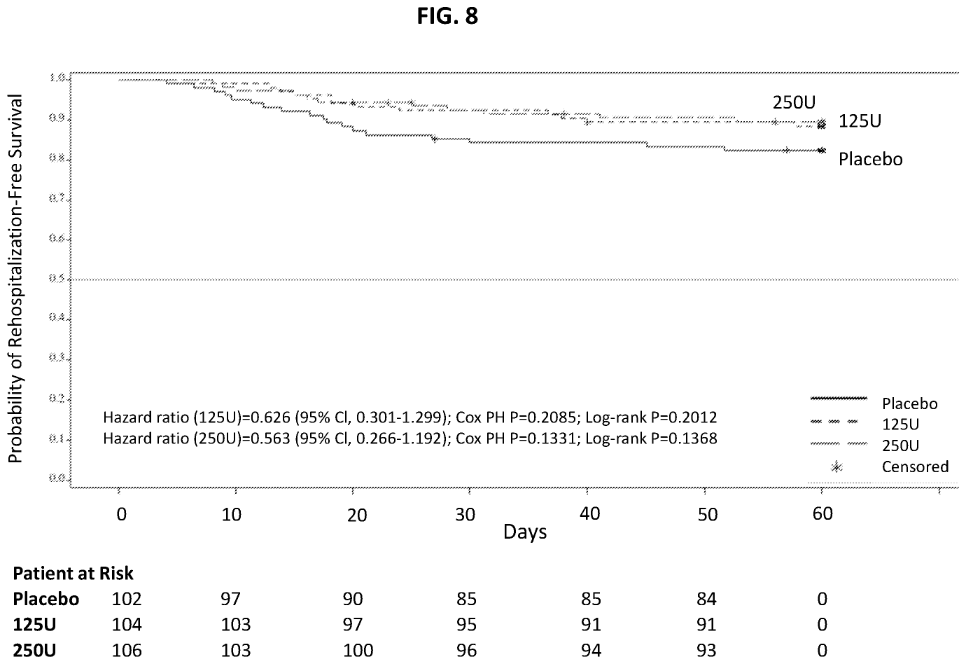

As shown in FIG. 7 and Table 10, all-cause 30-day rehospitalization occurred in 16 patients (15.7%) of the placebo group, 9 patients (8.7%) of the 125 U group (RR vs placebo 0.55, [95% CI 0.26, 1.19], P=0.13), and 10 patients (9.4%) in the 250 U group (RR vs placebo 0.60, [95% CI 0.29, 1.27], P=0.18). The Kaplan Meier curves of time to first rehospitalization during the first 60 days post-surgery for all cause hospitalization in all patients shown in FIG. 8 demonstrated a hazard ratio of 0.63 (p=0.2) for the 125 U group and 0.56 (p=0.14) for the 250 U group compared to the placebo group. Similar to the trend observed in all patients, the proportion of rehospitalization occurrence in the ≥65 years old and no-valve surgery subgroups were also lower among patients injected with the toxin at both doses (Table 10). These data suggest that toxin administration as described herein have surprising long-term benefits and can reduce clinical AF burden, such as all-cause rehospitalization, for all patients.

TABLE 10
All-cause rehospitalization by ≥65 years old and no-valve surgery subgroups

| Endpoint | Placebo | 125 U | 250 U |
|---|---|---|---|
| Overall | (N = 102) | (N = 104) | (N = 106) |
| All-cause Rehospitalization, w/in 30 days | 16 (15.7) | 9 (8.7) | 10 (9.4) |
| discharge, n (%) | | | |
| Relative Risk vs. Placebo (95% CI) | | 0.55 (0.26, 1.19) | 0.60 (0.29, 1.27) |
| p-value | | 0.1263 | 0.1775 |
| Age ≥65 years | (N = 61) | (N = 63) | (N = 61) |
| All-cause Rehospitalization, w/in 30 days | 10 (16.4) | 4 (6.3) | 5 (8.2) |
| discharge, n (%) | | | |
| Relative Risk vs. Placebo (95% CI) | | 0.39 (0.13, 1.17) | 0.49 (0.18, 1.36) |
| p-value | | 0.0790 | 0.1625 |
| Isolated CABG | (N = 65) | (N = 67) | (N = 67) |
| All-cause Rehospitalization, w/in 30 days | 10 (15.4) | 5 (7.5) | 3 (4.5) |
| discharge, n (%) | | | |
| Relative Risk vs. Placebo (95% CI) | | 0.48 (0.17, 1.34) | 0.29 (0.09, 1.02) |
| p-value | | 0.1539 | 0.0380 |

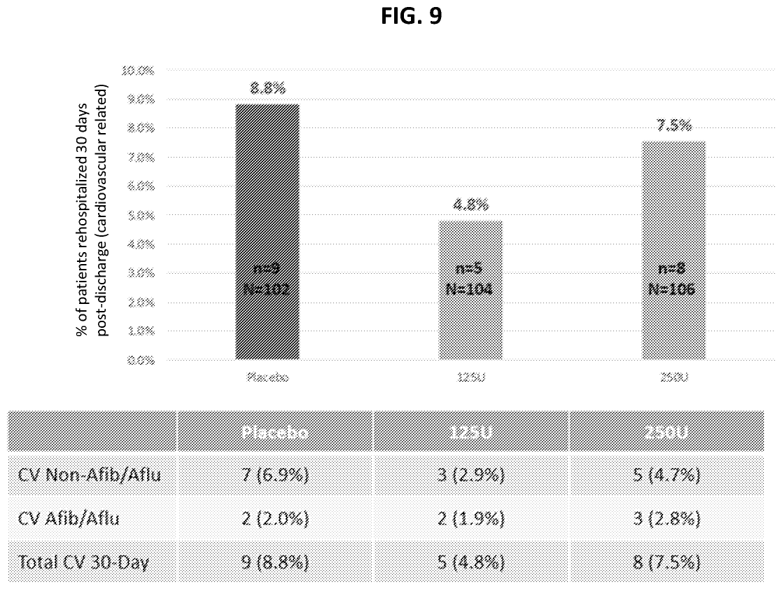

In particular, as shown in FIG. 9, the numbers of patients rehospitalized due to cardiovascular conditions 30 days post-discharge, were 9 patients (8.8%) in the placebo group, 5 patients (4.8%) in the 125 U group, and 8 patients (7.5%) in the 250 U group. These results are consistent with the lower rate of POAF in the 125 U group in all patients as described in Example 2.

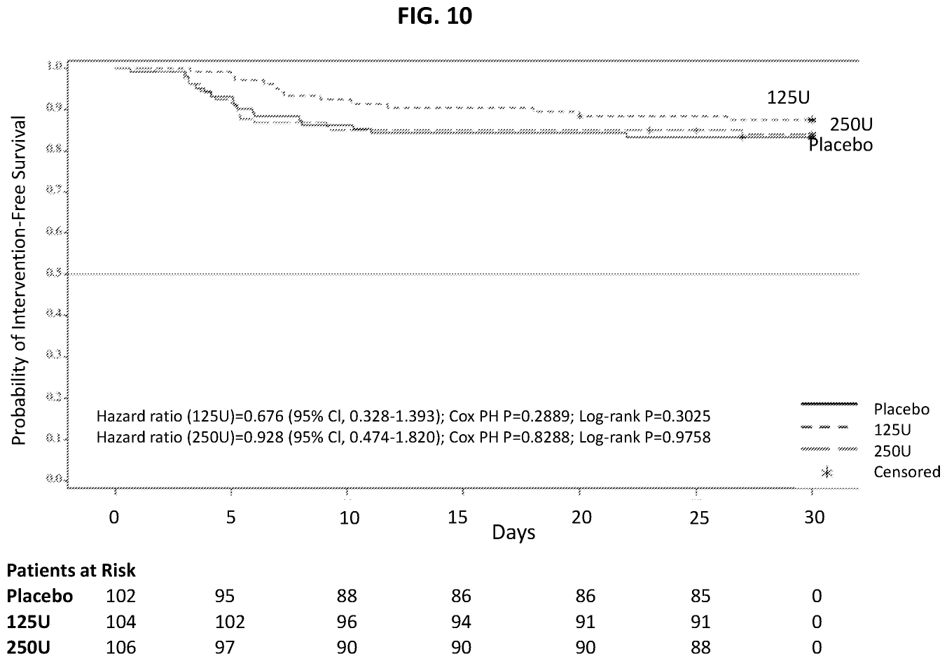

The number of participants free from AF through 12 months and off oral anticoagulation at the last study visit was 32 patients (45.1%) in the placebo group, 44 patients (62.9%) in the 125 U group (p=0.03 vs placebo), and 34 patients (47.2%) in the 250 U group (p=0.71 vs placebo). Time to anticoagulation was also favorable for the 125U group vs placebo. As shown in FIG. 10, administration of 125 U gemibotulinumtoxinA in all patients reduced anticoagulant usage by 32.4% (hazard ratio=0.676).

Given the role of inflammation in the propagation of atrial fibrillation post-surgery, biomarkers of inflammation were also evaluated at baseline and post-surgery. Surprisingly, fold change (FC) from baseline of mean IL-6 serum concentrations on Day 2 was 11.46 in 125 U vs 14.49 in placebo (FC ratio 0.83, SE 0.07, P=0.04). In CABG-only patients, FC for IL-6 from baseline on Day 2 was 10.81 in 125 U vs 15.87 in placebo (FC ratio 0.69, SE 0.08, P=0.0013) and 11.79 in 250U (FC ratio 0.79, SE 0.09, P=0.04). Surprisingly, serum concentrations of hs-CRP continued to increase from baseline up to Day 3 in placebo (FC 127.4) vs FC of 101.5 in 125 U and 93.27 in 250 U on Day 3. In CABG-only patients, sequential reductions of serum hs-CRP levels were observed between Days 2 to 4 among the placebo, 125 U and 250 U with the highest 250 U dose demonstrating lower FC on Day 2 (FC ratio 0.78, SE 0.08, p=0.02) and Day 4 (FC ratio 0.78, SE 0.09, P=0.02).

The rates of any adverse effects (AE) were similar in the 3 treatment groups: 93 (88.6%) with placebo, 92 (87.6%) with 125 U, and 104 (95.4%) with 250 U. Rates of AEs leading to discontinuation of study were also similar in the 3 treatment groups: 1 (1%) with placebo, 0 with 125 U, and 2 (1.8%) with 250 U. Overall, 8 patients underwent cardiac pacemaker implantation: 1 in the placebo group, 2 in the 125 U group, and 5 in the 250 U group. There were 2 deaths in those treated with 250 U (n=1 aorto-esophageal fistula and n=1 cardiac arrest). Pulmonary function testing revealed no statistically significant difference between the 3 groups.

### Example 7: Injection of Toxin to Epicardial Fat Pads During Cannula Placement and while on Bypass

A cardiac surgery was performed on a patient that involved cardiopulmonary bypass. In order to reduce bypass time, botulinum toxin was injected to the aortic fat pad while the cannulas were being inserted. The timing of this injection was not problematic and helped to decrease bypass time. The pulmonary vein fat pads were then injected while the patient was on bypass with approximately 3-4 minutes needed. The total injection time into the five epicardial fat pads was about 5 minutes.

### Example 8: Injection of Toxin to Aortic Fat Pads with Needle Bent Before Initiation of Cardiopulmonary Bypass

A patient undergoing cardiac surgery was not yet on cardiopulmonary bypass. The aortic fat pad was identified and noticed that it was very thin (i.e., 1 mm or less). In order to appropriately deposit the 1 mL of botulinum toxin, the needle was inserted into tissue at an angle of approximately 60-80 degrees before initiation of cardiopulmonary bypass (i.e., before the bypass cannulas are inserted). This approach made the injection of this fat pad much quicker, as the needle could clearly be seen under the epicardial fat, with no concern of puncturing the aorta. After the aortic fat pad was injected, the pulmonary vein fat pads were injected while the patient was on bypass. The time for injection into the four pulmonary vein fat pads was approximately 3-4 minutes. The total time for injection into the five epicardial fat pads was approximately 5 minutes.

### Example 9: Injection of Toxin to Epicardial Fat Pads During Bypass and Guided by Forceps

A patient undergoing cardiac surgery was on cardiopulmonary bypass. An amount of botulinum toxin was administered first to the aortic fat pad via injection. Subsequent to this, an amount of botulinum toxin was administered via injection to each of the two right-side pulmonary vein fat pads. It was noted that the 3 mL syringe with ½ inch needle had some difficulty with reaching the targets. In order to better guide the needle, forceps were used to grasp the lower part of the syringe (i.e., the region of the syringe adjacent to the needle) to better direct the injection. Once the needle successfully punctured the target, the plunger was pushed to administer the toxin. Injections were also made into the left-side pulmonary vein fat pads using forceps.

### Example 10: Identification of Successful Injection

A patient undergoing cardiac surgery was on cardiopulmonary bypass. An amount of botulinum toxin was administered first to the aortic fat pad via injection. Subsequent to this, an amount of botulinum toxin was administered via injection to each of the two right-side pulmonary vein fat pads and then to each of the two left-side pulmonary vein fat pads. A clear wheal or swelling of the epicardial fat tissue was observed, which indicated that the target was successfully injected.

### Example 11: Injection of Toxin to Pulmonary Vein Fat Pads with a Longer Needle

A patient undergoing cardiac surgery was noted as having a “barrel chest.” The left-side pulmonary vein fat pads were very deep in the chest and was not readily accessible with a half inch needle, even with the use of forceps. A longer needle was utilized (in this specific case a spinal needle) in order to effectively inject these left-side pulmonary vein fat pads.

Another patient undergoing cardiac surgery was noted as having a “small opening” in the chest after the sternotomy was performed. This “small opening” limited the amount of cardiac manipulation (i.e., shifting the heart to the right or left) in order to access the pulmonary vein fat pads. In this case, both the right-side and left-side pulmonary vein fat pads were injected using a spinal needle. In addition, the spinal needle was bent approximately 30 degrees, in order to curve around the heart, which allowed the needle to reach the pulmonary vein fat pads.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method for preventing post-operative atrial fibrillation (POAF) in a subject in need thereof, comprising:
selecting a subject in need of open-chest coronary artery bypass grafting (CABG) surgery and that is not in need of cardiac valve surgery,
administering to five epicardial fat pads of the subject about 125 units of a botulinum toxin serotype A by injection while the subject is undergoing open-chest CABG surgery, wherein the five epicardial fat pads comprise an aortic fat pad, a superior right-side pulmonary vein fat pad, an anterior right-side pulmonary vein fat pad, a superior left-side pulmonary vein fat pad, and an anterior left-side pulmonary vein fat pad,
wherein the subject does not exhibit at least one continuous post-operative atrial fibrillation episode having a duration of 30 seconds or more during a period of 30 days from administration,
thereby preventing post-operative atrial fibrillation in the subject.

2. The method of claim 1, further comprising selecting a subject that is about 65 years of age or older.

3. The method of claim 1 or 2, further comprising selecting a subject that is about 70 years of age or older.

4. The method of any one of the previous claims, wherein, during a period of 30 days from administration, the subject does not exhibit at least one continuous post-operative atrial fibrillation episode having a duration selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours.

5. The method of claim 4, wherein, during a period of 30 days from administration, the subject does not exhibit at least one continuous post-operative atrial fibrillation episode having a duration equal to or greater than 5 minutes.

6. The method of claim 4, wherein, during a period of 30 days from administration, the subject does not exhibit at least one continuous post-operative atrial fibrillation episode having a duration equal to or greater than 30 minutes.

7. The method of claim 4, wherein, during a period of 30 days from administration, the subject does not exhibit at least one continuous post-operative atrial fibrillation episode having a duration equal to or greater than 1 hour.

8. The method of claim 4, wherein, during a period of 30 days from administration, the subject does not exhibit at least one continuous post-operative atrial fibrillation episode having a duration equal to or greater than 4 hours.

9. The method of any one of the previous claims, wherein administering the botulinum toxin serotype A to the subject reduces the occurrence of one or more post-operative atrial fibrillation episodes by at least 20% during a period of 30 days from administration, as compared to a subject that does not receive administration of the toxin serotype A while undergoing open-chest CABG surgery, and wherein the one or more atrial fibrillation episodes have a duration of 30 seconds or more.

10. The method of claim 9, wherein the one or more post-operative atrial fibrillation episodes have a duration selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours during a period of 30 days from administration.

11. The method of claim 9, wherein administering the botulinum toxin serotype A to the subject reduces the occurrence of one or more post-operative atrial fibrillation episodes by at least 30% during a period of 30 days from administration, as compared to a subject that does not receive administration of the toxin serotype A while undergoing open-chest CABG surgery, wherein the one or more atrial fibrillation episodes have a duration of 30 seconds or more.

12. The method of claim 11, wherein the one or more atrial fibrillation episodes have a duration selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours during a period of 30 days from administration.

13. The method of claim 9, wherein administering the botulinum toxin serotype A to the subject reduces the occurrence of one or more post-operative atrial fibrillation episodes by at least 40% during a period of 30 days from administration, as compared to a subject that does not receive administration of the toxin serotype A while undergoing open-chest CABG surgery, wherein the one or more atrial fibrillation episodes have a duration of 30 seconds or more.

14. The method of claim 13, wherein the one or more atrial fibrillation episodes have a duration selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours during a period of 30 days from administration.

15. The method of claim 9, wherein administering the botulinum toxin serotype A to the subject reduces the occurrence of one or more atrial fibrillation episodes by at least 50% during a period of 30 days from administration, as compared to a subject that does not receive administration of the toxin serotype A while undergoing open-chest CABG surgery, wherein the one or more atrial fibrillation episodes have a duration of 30 seconds or more.

16. The method of claim 15, wherein the one or more atrial fibrillation episodes have a duration selected from the group consisting of equal to or greater than 2 minutes, equal to or greater than 5 minutes, equal to or greater than 6 minutes, equal to or greater than 30 minutes, equal to or greater than 1 hour, equal to or greater than 4 hours, equal to or greater than 6 hours, equal to or greater than 12 hours, and equal to or greater than 24 hours during a period of 30 days from administration.

17. A method of reducing the occurrence of hospital readmission within 60 days after discharge after open-chest CABG surgery in a subject in need thereof, comprising:
selecting a subject in need of open-chest coronary artery bypass grafting (CABG) and that is not in need of cardiac valve surgery,
administering to five epicardial fat pads of the subject about 125 units of a botulinum toxin serotype A by injection while the subject is undergoing open-chest CABG surgery, wherein the five epicardial fat pads comprise an aortic fat pad, a superior right-side pulmonary vein fat pad, an anterior right-side pulmonary vein fat pad, a superior left-side pulmonary vein fat pad, and an anterior left-side pulmonary vein fat pad,
thereby reducing the occurrence of hospital readmission within 60 days after discharge after open-chest CABG surgery.

18. The method of claim 17, further comprising selecting a subject that is about 65 years of age or older.

19. The method of claim 17 or 18, further comprising selecting a subject that is about 70 years of age or older.

20. The method of any one of claims 17-19, wherein the administering reduces the occurrence of hospital readmission of the subject by at least 30% during a period of 30 days from discharge, as compared to a subject that does not receive administration of the toxin serotype A while undergoing open-chest CABG surgery.

21. The method of claim 20, wherein the administering reduces the occurrence of hospital readmission of the subject by at least 40% during a period of 30 days from discharge, as compared to a subject that does not receive administration of the toxin serotype A while undergoing open-chest CABG surgery.

22. The method of claim 20, wherein the administering reduces the occurrence of hospital readmission of the subject by at least 50% during a period of 30 days from discharge, as compared to a subject that does not receive administration of the botulinum toxin serotype A while undergoing open-chest CABG surgery.

23. The method of any one of the previous claims, further comprising selecting a subject that has a history of paroxysmal atrial fibrillation or a history of persistent atrial fibrillation.

24. The method of any one of claims 1-22, further comprising selecting a subject that does not have a history of paroxysmal atrial fibrillation or a history of persistent atrial fibrillation.

25. The method of any one of the previous claims, wherein the administering results in a reduction in length of stay of the subject in intensive care unit (ICU) by at least 8 hours, as compared to a subject that does not receive administration of the botulinum toxin serotype A while undergoing open-chest CABG surgery.

26. The method of any one of the previous claims, wherein the administering results in a reduction in length of hospital stay of the subject by at least 1 day, as compared to a subject that does not receive administration of the botulinum toxin serotype A while undergoing open-chest CABG surgery.

27. The method of any one of the previous claims, wherein the administering reduces anticoagulant usage for the subject by at least 30% during a period of 30 days from administration, as compared to a subject that does not receive administration of the botulinum toxin serotype A while undergoing open-chest CABG surgery.

28. The method of claim 27, wherein the administering reduces anticoagulant usage for the subject by at least 40% during a period of 30 days from administration, as compared to a subject that does not receive administration of the botulinum toxin serotype A while undergoing open-chest CABG surgery.

29. The method of any one of the previous claims, wherein the subject has received a beta-blocker therapy before the open-chest CABG surgery and is withdrawn from the beta-blocker therapy after the open-chest CABG therapy.

30. The method of claim 29, wherein the administering of the botulinum toxin serotype A to the subject withdrawn from the beta-blocker therapy reduces the occurrence of one or more post-operative atrial fibrillation episodes by at least 20% during a period of 30 days from administration, as compared to a subject that does not receive administration of the toxin serotype A while undergoing open-chest CABG surgery, and wherein the one or more atrial fibrillation episodes have a duration of 30 seconds or more.

31. The method of claims 29 and 30, wherein the administering reduces the occurrence of one or more atrial fibrillation episodes by at least 40% during a period of 30 days from administration, as compared to a subject that does not receive administration of the toxin serotype A while undergoing open-chest CABG surgery, wherein the one or more atrial fibrillation episodes have a duration of 30 seconds or more.

32. The method of claim 31, wherein the administering reduces the occurrence of one or more post-operative atrial fibrillation episodes by at least 50% during a period of 30 days from administration, as compared to a subject that does not receive administration of the toxin serotype A while undergoing open-chest CABG surgery, wherein the one or more atrial fibrillation episodes have a duration of 30 seconds or more.

33. The method of any one of the previous claims, wherein the administering comprises administering about 10 units to about 40 units of the botulinum toxin serotype A to each of the five epicardial fat pads, for a total dose of about 125 units.

34. A method of reducing anticoagulant usage after open-chest CABG surgery in a subject in need thereof, comprising:
selecting a subject in need of open-chest coronary artery bypass grafting (CABG) and that is not in need of cardiac valve surgery,
administering to five epicardial fat pads of the subject about 125 units of a botulinum toxin serotype A by injection while the subject is undergoing open-chest CABG surgery, wherein the five epicardial fat pads comprise an aortic fat pad, a superior right-side pulmonary vein fat pad, an anterior right-side pulmonary vein fat pad, a superior left-side pulmonary vein fat pad, and an anterior left-side pulmonary vein fat pad,
wherein the administering reduces anticoagulant usage by at least 20% during a period of 30 days from administration, as compared to a subject that does not receive administration of the toxin serotype A while undergoing open-chest CABG surgery.

35. The method of claim 34, wherein reducing anticoagulant usage comprises delaying the time it takes to first anticoagulant use, reducing the amount of anticoagulant used, or both, during a period of 30 days after open-chest CABG surgery.

36. The method of claim 35, wherein the administering reduces anticoagulant usage for the subject by at least 30% during a period of 30 days from administration, as compared to a subject that does not receive administration of the botulinum toxin serotype A while undergoing open-chest CABG surgery.

37. The method of claim 35, wherein the administering reduces anticoagulant usage for the subject by at least 40% during a period of 30 days from administration, as compared to a subject that does not receive administration of the botulinum toxin serotype A while undergoing open-chest CABG surgery

38. The method of any one of the previous claims, wherein the administering comprises administering about 20 units to about 30 units of the botulinum toxin serotype A to each of the five epicardial fat pads, for a total dose of about 125 units.

39. The method of claim 38, wherein the administering comprises administering about 25 units of the botulinum toxin serotype A to each of the five epicardial fat pads, for a total dose of about 125 units.

40. The method of claim 39, wherein the administering comprises injecting 1 mL of a botulinum toxin serotype A solution having a concentration of about 25 units/mL into each of the five epicardial fat pads.

41. The method of any one of the previous claims, wherein the botulinum toxin serotype A is animal protein free.

42. The method of any one of the previous claims, wherein the botulinum toxin serotype A is selected from daxibotulinumtoxinA, nivobotulinumtoxinA, and gemibotulinumtoxinA.

43. The method of claim 42, wherein the botulinum toxin serotype A is gemibotulinumtoxinA.

44. The method of claim 43, wherein gemibotulinumtoxinA is provided in a lyophilized formulation comprising trehalose, poloxamer P188, L-methionine and histidine.

45. The method of claim 43, wherein gemibotulinumtoxinA is provided in a solution comprising trehalose, poloxamer P188, L-methionine and histidine.

46. The method of any one of claims 1-41, wherein the botulinum toxin serotype A is a pure toxin.

47. The method of claim 46, wherein the botulinum toxin serotype A is incobotulinumtoxinA.

48. The method of claim 46, wherein the botulinum toxin serotype A is daxibotulinumtoxinA.

49. The method of any of the previous claims, wherein the botulinum toxin serotype A is not co-administered with a second agent selected from the group consisting of: a drug that interferes with neuromuscular transmission, an aminoglycoside, an anticholinergic drug, or a muscle relaxant.

50. The method of any one of the previous claims, wherein the administering comprises injecting the botulinum toxin at 1-5 locations per fat pad.

51. The method of claim 50, wherein the administering comprises injecting the botulinum toxin at 1, 2 or 3 locations per fat pad.

52. The method of any one of the previous claims, wherein the time for administering is about 5-30 seconds per injection, or 5-150 seconds per fat pad.

53. The method of claim 52, wherein the time for administering is about 10-15 seconds per injection, or 10-45 seconds per fat pad.

54. The method of any one of the previous claims, wherein injections are made at a depth of about 1-2 mm.

55. The method of any one of the previous claims, wherein injections are made at an oblique angle.

56. The method of any one of the previous claims, wherein the administering comprises injecting the botulinum toxin at one location of each of the superior and anterior right-side pulmonary vein fat pads and the superior and anterior left-side pulmonary vein fat pads.

57. The method of any one of the previous claims, wherein the botulinum toxin is first administered to the aortic fat pad.

58. The method of any one of the previous claims, wherein the method does not comprise electrically stimulating the epicardial fat pads to locate an administration site on the epicardial fat pads.

59. The method of any one of the previous claims, wherein the administering comprises administering the botulinum toxin to the epicardial fat pads in a sequential order of the aortic fat pad, the superior and anterior right-side pulmonary vein fat pads, and the superior and anterior two left-side pulmonary vein fat pads.

60. The method of any one of claims 1-58, wherein the administering comprises administering the effective amount of botulinum toxin to the epicardial fat pads in a sequential order of the aortic fat pad, the superior and anterior left-side pulmonary vein fat pads, and the superior and anterior two right-side pulmonary vein fat pads.

61. The method of claim 59 or claim 60, wherein administering in the sequential order reduces time for administering the effective amount to the epicardial fat pads relative to administering the effective amount in an order other than the sequential order.

62. The method of any one of the previous claims, wherein the administering provides reduced risk of leakage and tissue trauma.

63. The method of any one of the previous claims, wherein the administering is performed after pericardial sac is dissected and before the primary surgical procedure.

64. The method of claim 63, wherein the administering comprises administering (i) before cardiopulmonary bypass is initiated, (ii) while bypass cannulas are being placed on the subject; (iii) while the subject is on cardiopulmonary bypass, (iv) after cross-clamping has been performed, and/or (v) after cardioplegia has been instituted.

65. The method of claim 64, wherein the administering comprises administering botulinum toxin into the aortic fat pad while the bypass cannulas are being inserted and into the pulmonary vein fat pads while the subject is on cardiopulmonary bypass.

66. The method of claim 64, wherein the administering comprises administering botulinum toxin into the aortic fat pad, the superior and anterior right-side pulmonary vein fat pads, and the superior and anterior left-side pulmonary vein fat pads while the subject is on cardiopulmonary bypass.

67. The method of claim 64, wherein the administering comprises administering botulinum toxin into the aortic fat pad before cardiopulmonary bypass is initiated and into the pulmonary vein fat pads while the subject is on cardiopulmonary bypass.

68. The method of claim 63, wherein the subject is on off-pump bypass and wherein the botulinum toxin is administered when the subject is undergoing off-pump bypass surgery.

69. The method of any one of the previous claims, wherein the administering comprises injecting botulinum toxin via a syringe to the aortic fat pad wherein the syringe has a needle bent at an angle ranging from 10 to 90 degrees.

70. The method of claim 69, wherein upon insertion of the needle into the aortic fat pad, the syringe is drawn back to ensure the needle is not inside the aorta.

71. The method of any one of the previous claims, wherein the administering comprises injecting botulinum toxin via a syringe to each of the superior and anterior right-side pulmonary vein fat pads wherein the syringe has a needle bend at an angle ranging from 10 to 50 degrees.

72. The method of claim 71, wherein upon insertion of the needle into one or both of the superior and anterior right-side pulmonary vein fat pads, the syringe is drawn back to ensure the needle is not inside the pulmonary vein or atria.

73. The method of claim 71, wherein the injecting via a syringe to each of the superior and anterior right-side pulmonary vein fat pads wherein the syringe has a needle bend at an angle ranging from 10 to 50 degrees allows for quicker deposits of the botulinum toxin and reduces the need for manipulation of the heart.

74. The method of any one of the previous claims, wherein the administering comprises injecting botulinum toxin via a syringe to each of the superior and anterior left-side pulmonary vein fat pads, wherein the syringe has a needle, and wherein the needle of the syringe is bent at an angle ranging from 10 to 50 degrees.

75. The method of claim 74, wherein upon insertion of the needle into one or both of the superior and anterior left-side pulmonary vein fat pads, the syringe is drawn back to ensure the needle is not inside the pulmonary vein or atria.

76. The method of any one of claims 69-75, wherein needle gauge ranges from 25 to 30.

77. The method of any one of claims 69-76, wherein the needle length of the syringe ranges from 0.5 to 3.5 inch.

78. The method of any one of claims 69-77, wherein the needle is a spinal needle.

79. The method of claim 78, wherein the spinal needle is used to administer the botulinum toxin to the superior and anterior left-sided pulmonary vein fat pads.

80. The method of any one of claims 69-79, wherein forceps are used to guide the needle.
